(19) United States
(12) Patent Application Publication
(10) Pub. No.: US 2016/0012011 A1
(43) Pub. Date: Jan. 14, 2016

(54) PRECISE FREQUENCY-PATTERN ANALYSIS TO DECOMPOSE COMPLEX SYSTEMS INTO FUNCTIONALLY INVARIANT ENTITIES

(71) Applicant: NEW YORK UNIVERSITY, New York, NY (US)

(72) Inventors: Rodolfo Llinas, New York, NY (US); Mikhail N. Ustinin, Moscow (RU)

(73) Assignee: NEW YORK UNIVERSITY, New York, NY (US)

(21) Appl. No.: 14/795,507

(22) Filed: Jul. 9, 2015

Related U.S. Application Data
(60) Provisional application No. 62/022,405, filed on Jul. 9, 2014.

(51) Int. Cl. G06F 17/14; A61B 5/04; A61B 5/0402

(52) U.S. Cl. CPC G06F 17/14; A61B 5/0402; A61B 5/04008

(57) ABSTRACT

Methods, systems, apparatuses, including computer programs encoded on computer readable media, for registering a multichannel time series from data collected from a plurality of channels monitoring a body. Each channel corresponds to one or more sensors. The data from all channels is precise Fourier transformed to frequency data in a frequency domain. The frequency data is inverse Fourier transformed for each frequency in the frequency domain. Elementary coherent oscillations are determined from the inverse Fourier transformed frequency data. Partial spectra of the functional entities include frequencies with similar patterns from the elementary coherent oscillations. A functional tomogram of the body is calculated from the distribution in space of the energy and/or dominant directions of the sources, producing the partial spectra. The partial spectra is inverse Fourier transformed to reconstruct a time series for the functional entities.

### CROSS REFERENCE TO RELATED APPLICATIONS

This application claims the benefit of U.S. Provisional Patent Application No. 62/022,405, filed Jul. 9, 2014, which is incorporated by reference herein in its entirety. This application is also related to U.S. patent application Ser. No. 14/055,692, filed Oct. 16, 2013, which is incorporated by reference herein in its entirety.

### BACKGROUND

Multichannel measurements have greatly increased understanding of complex systems in various fields of knowledge, such as astronomy, geological physics etc., and are producing large fundamental and applied impacts. Registration of the dynamic signals by spatially distributed sensors is aimed on the functional reconstruction of the system under study. Analysis of the registered data must allow estimating spatial structures and time behavior of the objects composing the system. There are many important applications of multichannel measurements in biology and medicine, such as electric cardiography, magnetic cardiography, vectorcardiography, electric encephalography etc. Another example is magnetic encephalography (MEG) that allows the study of brain activity.

MEG records magnetic fields that are produced by electrical currents occurring in the brain, using numerous external magnetometers. There are various advantages of using MEG, as it is non-invasive, highly sensitive, has a high sampling rate and many channels of registration. However, MEG is not without its challenges, like many other multichannel methods. The complicated configuration of brain activity sources, which can be working simultaneously, can make localization of brain activity sources difficult. Further, there is a large amount of data generated and as the magnetic fields produced in the brain are relatively small, the sensing of the magnetic fields is susceptible to external and/or internal noise.

Current methods of processing MEG data are rough and approximate. For example, using Fourier Transforms that use a short time window leads to a rough frequency grid. That is, the MEG at one frequency can unite many activities and noises that are close in frequency. This can lead to problems when trying to localize the sources using an Inverse Fourier Transform.

### SUMMARY

In general, one aspect of the subject matter described in this specification can be embodied in methods for registering a multichannel time series from data collected from a plurality of channels monitoring a body. Each channel corresponds to one or more sensors. The data from all channels is precise Fourier transformed to frequency data in a frequency domain. The frequency data is inverse Fourier transformed for each frequency in the frequency domain. Elementary coherent oscillations are determined from the inverse Fourier transformed frequency data. Partial spectra of the functional entities include frequencies with similar patterns from the elementary coherent oscillations. A functional tomogram of the body is calculated from the distribution in space of the energy and/or dominant directions of the sources, producing the partial spectra. The partial spectra is inverse Fourier transformed to reconstruct a time series for the functional entities. Other implementations of this aspect include corresponding systems, apparatuses, and computer-readable media configured to perform the actions of the method.

The foregoing summary is illustrative only and is not intended to be in any way limiting. In addition to the illustrative aspects, implementations, and features described above, further aspects, implementations, and features will become apparent by reference to the following drawings and the detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are, therefore, not to be considered limiting of its scope, the disclosure will be described with additional specificity and detail through use of the accompanying drawings.

FIG. 1 illustrates a system for localizing a signal within a body in accordance with an illustrative implementation.

FIG. 2 is a flow diagram depicting operations for localizing a signal within a body in accordance with an illustrative implementation.

FIG. 3 illustrates spacing of sensors in a heart monitoring experiment in accordance with an illustrative implementation.

FIG. 4 illustrates a magnetic cardiogram multichannel spectrum in accordance with an illustrative embodiment.

FIGS. 5 and 6 illustrate a comparison between MCG and ECG data in accordance with an illustrative embodiment.

FIGS. 7A-7G illustrate the precise frequency-pattern analysis and coherence analysis for MCG data in accordance with an illustrative embodiment.

FIG. 8 illustrates a precise Fourier spectrum for ECG data in accordance with an illustrative embodiment.

FIG. 9 is a graph of an alpha-rhythm peak in the general spectrum.

FIGS. 10-12 illustrate a user interface to analyze alpha-rhythm peaks in accordance with an illustrative embodiment.

FIG. 13 illustrates two oscillations that are phase shifted.

FIGS. 14A and 14B illustrate the inverse problem solution for two independent coherent signals in accordance with an illustrative embodiment.

FIG. 15 illustrates a MEG 3-dimensional spectrum in accordance with an illustrative implementation.

FIG. 16 illustrates devices used to measure magnetic fields of a subject in accordance with an illustrative implementation.

FIG. 17 is a graph of a stimulus profile used in an MEG experiment in accordance with an illustrative implementation.

FIG. 18 is a graph of harmonics for a stimulus in accordance with an illustrative implementation.

FIGS. 19A-19C illustrate the optimization of a stimulus spectrum in accordance with an illustrative implementation.

FIGS. 20A-20E illustrate the detailed analysis of the response multichannel spectrum at the stimulus harmonics in accordance with an illustrative implementation.

FIG. 21 is a graph of precisely tuned multichannel spectrum of measured magnetic fields in accordance with an illustrative implementation.

FIG. 22 is a graph of precisely tuned multichannel spectrum of measured magnetic fields in a narrow frequency range that includes the 8^{th} harmonic of the stimulus and the power grid spectrum in accordance with an illustrative implementation.

FIG. 23 is a graph of precisely tuned multichannel spectrum of measured magnetic fields in a narrow frequency range that includes the 8^{th} harmonic of the stimulus in accordance with an illustrative implementation.

FIG. 24 is a graph that illustrates phase shifts between channels at the frequency of the 8^{th} harmonic of the stimulus in accordance with an illustrative implementation.

FIG. 25 illustrates a noise magnetic field from a power grid in accordance with an illustrative implementation.

FIG. 26 is a flow diagram for spontaneous field analysis in accordance with an illustrative implementation.

FIG. 27 is a flow diagram for evoked field analysis in accordance with an illustrative implementation.

FIG. 28 is a flow diagram for spontaneous field analysis in accordance with an illustrative implementation.

FIG. 29 is a graph of the MEG multichannel spectrum from 1.5 to 15 Hz of a test subject with thalamo-cortical dysrhythmia in accordance with an illustrative implementation.

FIG. 30 illustrates a location and directions of a trial dipole in accordance with an illustrative implementation.

FIG. 31 shows four normalized patterns, produced by a trial dipole in accordance with an illustrative implementation.

FIG. 32 illustrates a reconstruction of the time dependence of magnetic fields from a partial spectrum and the inverse problem solution to estimate spatial structure in accordance with an illustrative implementation.

FIG. 33 shows the distribution of the total energy between different directions of a trial dipole in accordance with an illustrative implementation.

FIG. 34 shows the general view of the multichannel spectrum in this band, giving the total decomposition of the MEG to functionally invariant entities in accordance with an illustrative implementation.

FIG. 35 shows the elementary block of the functional tomogram and the directions of the trial dipoles in accordance with an illustrative implementation.

FIG. 36 shows a functional tomogram and an MRI of the anatomical structure of a subject's head in the same experiment in accordance with an illustrative implementation.

FIG. 37 illustrates a selection of the brain and a set of sources, corresponding to this selection in accordance with an illustrative implementation.

FIG. 38 illustrates a functional tomogram of a brain, neck muscles, and olfactory system in the frequency band 0.003-100 Hz with spatial resolution 3 mm in accordance with an illustrative implementation.

FIG. 39 illustrates a functional tomogram of different frequency bands in accordance with an illustrative implementation.

FIG. 40 illustrates a functional tomogram of the human heart in the frequency band 1-12 Hz in accordance with an illustrative implementation. X, Y, Z in millimeters.

FIG. 41 illustrates three cycles of the magnetic cardiogram restored from the partial spectrum of the heart structure, shown in FIG. 40, in accordance with an illustrative implementation.

FIG. 42 illustrates a photography of the human hand and its functional tomogram, calculated from multichannel registration of magnetic field in accordance with an illustrative implementation.

FIG. 43 is a flow diagram for generating a functional tomographical map in accordance with an illustrative implementation.

FIG. 44 is a block diagram of a computer system in accordance with an illustrative implementation.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

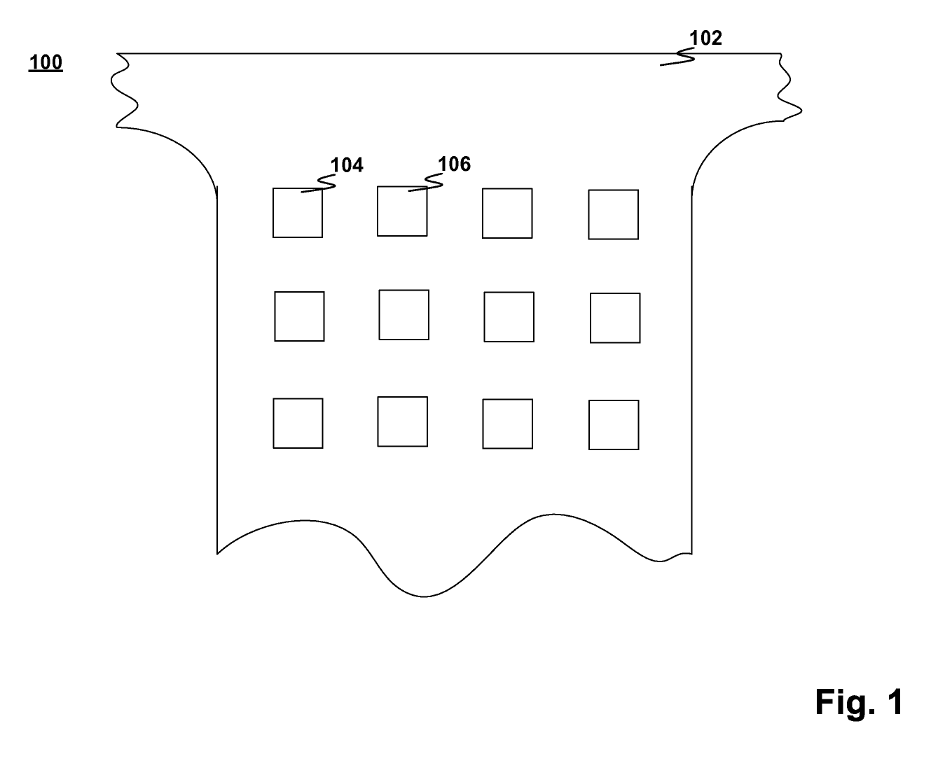

Recorded signals from multiple sensors placed on or near a body can be used to localize a signal within the body. FIG. 1 illustrates a system for localizing a signal within a body 102 in accordance with an illustrative implementation. The body 100 can be a constant spatial structure that includes a number of components located within the body 100. The body 100 can be any structure that contains components, such as, but is not limited to, portions of a human body, portions of an animal, a mechanical device, portions of a planet, enclosures containing fluids and/or gases, etc. Practically, any solid can be used as the body 100. The components are generally in a constant position and orientation while the signals are recorded. Although some movement, such as expansion and contraction, of the components are allowed. For example, a heart can be a component of the body 100, which has a constant position and orientation, but is not a constant size. A number of sensors, e.g., 104 and 106, are distributed over the body 100 and record signals from the body 100. A signal is generated by the components, either spontaneously (active media) or evoked by a stimulus (responding media). The signal evoked by the source components must be additive. Each sensor, e.g., 104 and 106, registers the sum of signals, coming from all sources, during a recording time T. As described in greater detail below, the recorded signals can then be used to localize a signal within the body 100. In other words, the spatial structure of the source of a signal can be determined. The spatial structure can be referred to as a functionally invariant entity since it produces the same signal pattern, e.g., normalized pattern as described below, during the recording time. In addition, using signals recorded over the recording time T, the time dependence of a signal sources can be determined.

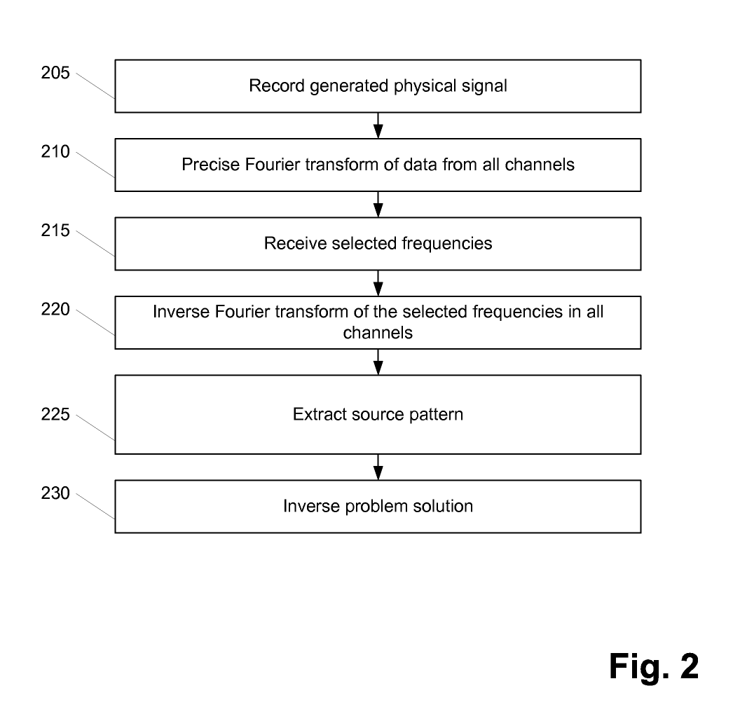

FIG. 2 is a flow diagram depicting operations for localizing a signal within a body in accordance with an illustrative implementation. Once any number of sensors have been placed on or near a body, each sensor can record signals detected in body over a time period. In an operation 205, K number of sensors record signals from all sources over a recording time T. The recorded signals include a generated signal within the body as well as other signals from the body. The recorded signals can be described by the following set of functions:

{\tilde{B}ᵢ(t)},i=1, . . . K,tε[0,T].

Using the above set of function, the spatial structure of the components of the body can be estimated. In addition, the time dependencies of the signals, generated by the components, can be estimated. In an operation 210, a precise Fourier transform of the data from each sensor is calculated. This operation essentially splits the signal in each channel (e.g., from each sensor) to a detailed representation of the signals in frequency space. After the transformation into frequency space, one or more frequencies can be selected for further analysis. In one implementation, the frequencies are selected using a user interface. In an operation 215, the selected frequencies are received from the user interface. An inverse Fourier transform can be done on the selected frequencies in an operation 220. In one implementation, the inverse Fourier transformation of a selected frequency vₙ in all channels provides the following set of functions:

$$\{ {B}_{ni} ( t ) \} ; i = 1 , K , t ∈ [ 0 , {T}_{{v}_{n}} ] , {T}_{{v}_{n}} = \frac{1}{{v}_{n}} .$$

In an operation 225, a source pattern can be extracted using the above set of functions. If the above set of functions in all channels are coherent, the source pattern will be constant through the reconstructed time. This demonstrates the existence of distinct constant spatial structure at a given frequency of a component within the body.

The above discussion is independent of the particular body. Accordingly, in order to determine the spatial structure producing the pattern, the characteristics of the given physical model of the body can be investigated. The inverse problem relating to the characteristics of the physical model of the body can be solved. In an operation 230, the inverse problem is solved. This solution determines the spatial structure of the component that generated the signal at vₙ.

Components within a body and/or the body itself can product their own signals (spontaneous activity). These spontaneous signals can be recorded by the sensors and analyzed as described above. In addition to spontaneous activity, forced signals can be produced by components within the body and/or the body itself. Forced signals are triggered by an external stimulus. Analysis of these signals can uncover many frequencies that give highly coherent oscillation patterns. These patterns can correspond to reasonable and interpretable inverse problem solutions. The inverse problem solutions allow the study of spatial structures contained within a body and to reconstruct the time course of signals through the body. Examples of analyzing both types of signals are described in greater detail below.

### Frequency-Time Analysis and Coherence

Once the sensors have recorded signals over a period of time, the data from each sensor can be analyzed. A signal measured at a sensor number i, can be written as:

$${\tilde{B}}_{i} ( t ) = {\sum }_{j = 1}^{M} {P}_{ij} {A}_{j} ( t ) ,$$

where Aⱼ(t) is a time dependence of the source number j, and matrix element Pᵢⱼ is given by the sensing character of the sensor number i in relation to the source number j.

The multichannel precise Fourier transform calculates a set of spectra for experimentally measured functions \tilde{B}ᵢ(t):

$${a}_{0 i} = \frac{2}{T} {\int }_{0}^{T} {\tilde{B}}_{i} ( t ) d t , {a}_{ni} = \frac{2}{T} {\int }_{0}^{T} {\tilde{B}}_{i} ( t ) cos ( 2 π {v}_{n} t ) d t , \\ {b}_{ni} = \frac{2}{T} {\int }_{0}^{T} {\tilde{B}}_{i} ( t ) sin ( 2 π {v}_{n} t ) d t ,$$

where a₀ᵢ, aₙᵢ, bₙᵢ, are Fourier coefficients for the frequency vₙ in the channel number i, and n=, . . . ,N,N=vₘₐₓT, where vₘₐₓ is the highest desirable frequency.

The term “Precise” is used in three different senses here and is achieved by three distinct steps:

1. Precise calculation of the Fourier integrals. For every channel the experimental set of points is interpolated, converting it to a continuous function \tilde{B}ᵢ(t). Gaussian quadrature formulas are used to calculate integrals on any interval [0, T], in the registration scale.

2. Building all spectra for the registration time T, which is sufficient to reveal the detailed frequency structure of the system. The step in frequency is

$$Δ v = {v}_{n} - {v}_{n - 1} = \frac{1}{T} ,$$

thus frequency resolution is determined by the recording time. Moving or a fractional window of sufficient width T can be used to study time dependence of precise spectra.

3. Tuning of the frequency grid by cutting the interval of integration T to build the optimal approximation of the frequency selected. Tuning can be performed by little changes of the integration time T.

This precise transform leads to an accurate and reversible representation of time data in the frequency domain for each channel. As for the space domain data, “space” is determined by data recorded in many channels, having different positions with respect to the source. That is, if an accurate representation of time series for all channels are used, spatial characteristics of the signal can also be determined accurately.

Given a precise multichannel spectra it is possible to perform the inverse Fourier transform using:

$${B}_{i} ( t ) = \frac{{a}_{0 i}}{2} + {\sum }_{n = 1}^{N} {a}_{ni} cos ( 2 π {v}_{n} t ) + {\sum }_{n = 1}^{N} {b}_{ni} sin ( 2 π {v}_{n} t ) , \\ {v}_{n} = \frac{n}{T} , N = {v}_{max} T ,$$

where a₀ᵢ, aₙᵢ, bₙᵢ—are Fourier coefficients, found in the previous formula above.

This formula can also be written as

$${B}_{i} ( t ) = \frac{{a}_{0 i}}{2} + {\sum }_{n = 1}^{N} {ρ}_{ni} sin ( 2 π {v}_{n} t + {ϕ}_{ni} ) , {v}_{n} = \frac{n}{T} , N = {v}_{max} T ,$$

where ρₙᵢ=\sqrt{aₙᵢ²+bₙᵢ²}, φₙᵢ=atan 2(aₙᵢ, bₙᵢ). This transform allows the possibility of implementing precise filtering, including or eliminating any selected set of frequencies when restoring the signal. This does not limit the maximal frequency by the sampling grid. After the interpolation described above, all channels are continuous functions and can be approximated with any precision.

The basic idea of the precise frequency-pattern analysis is to study detailed frequency structure of the system, restoring multichannel signal at every frequency and analyzing the patterns obtained. In various implementations, the following function is used:

Bₙᵢ(t)=ρₙᵢ sin(2πvₙt+φₙᵢ)

A coherence definition can be determined by restoring the multichannel signal at particular frequency:

Bₙᵢ(t)=ρₙᵢ sin(2πvₙt+φₙᵢ),

where tε[0,Tᵥ_{ₙ}],

$${T}_{{v}_{n}} = \frac{1}{{v}_{n}}$$

is the period of this frequency.

The summary instantaneous power produced by all channels will be:

$${p}_{n} ( t ) = {\sum }_{i = 1}^{K} {B}_{ni}^{2} ( t ) .$$

Coherence of the sources on this frequency can be characterized by the value of phase coincidence, scaling from 1 to 0:

$${C}_{v} = 1 - \frac{min ( p ( t ) )}{max ( p ( t ) )} ,$$

Where min and max are calculated at the period Tᵥ_{ₙ}. Cᵥ, therefore, is a value between 0 and 1, inclusive. The physical sense of Cᵥ follows from Bₙᵢ(t)=ρₙᵢ sin(2πvₙt+φₙᵢ). Cᵥ is equal to 1, if all channels have equal phases at the frequency vₙ.

Invariant patterns can be extracted from the signals where there is a high coherence. This can be shown as followings.

Coherence Theorem 1.

The equality of phases in all channels is necessary and a sufficient condition for the invariance of pattern through the reconstructed time.

Coherence Theorem 2.

The equality of phases in all channels is necessary and sufficient condition for Cᵥ=1. This theorem provides a directly calculable feature to estimate the coherence at any frequency v.

Coherence Theorem 3.

The time course of a source, having arbitrary spatial structure, can be restored from the partial Fourier spectrum. This partial spectrum consists of the frequencies with coherence equal or close to 1, having the same normalized pattern. Spatial structure of the source can be found from this pattern.

Consider a coherent system, consisting of M sources, having the similar time dependencies:

Aⱼ(t)=cⱼA(t),

where cⱼ gives the force of source number j.

From

$${\tilde{B}}_{i} ( t ) = {\sum }_{j = 1}^{M} {P}_{ij} {A}_{j} ( t )$$

and Aⱼ(t)=cⱼA(t):

$${\tilde{B}}_{i} ( t ) = {\sum }_{j = 1}^{M} {P}_{ij} {A}_{j} ( t ) = {\tilde{P}}_{i} A ( t ) ,$$

where

$${\tilde{P}}_{i} = {\sum }_{j = 1}^{M} {P}_{ij} {c}_{j} .$$

It follows from the above formula that for every frequency

Bₙᵢ(t)=\tilde{P}ᵢρₙ sin(2πvₙt+φₙ),

where ρₙ sin(2πvₙt+φₙ)=Aₙ(t) is the n-th Fourier component of the function A(t).

The instantaneous power will be:

$${p}_{n} ( t ) = {ρ}_{n}^{2} {sin}^{2} ( 2 π {v}_{n} t + {ϕ}_{n} ) {\sum }_{i = 1}^{K} {\tilde{P}}_{i}^{2} ,$$

and has minimum=0 in the period.

A number of conclusions can be drawn from these theorems. For example, the coherent source of arbitrary form and common time course A(t) will give Cᵥ=1 for every frequency of function A(t). In addition, patterns at all frequencies of function A(t) will be identical; and the time course A(t) of the coherent source can be restored by the inverse Fourier transform performed over the spectra, combined from all frequencies with similar patterns. These theoretical considerations are the foundation for the reconstruction of time courses of functional entities with constant spatial structures, performing detailed frequency analysis and studying the similarity of the patterns with high coherence.

### Frequency-Pattern Analysis

In some cases, applying the above method to real data, the value of Cᵥ is less than 1. This can occur for various reasons, such as,
1) Non-correlated noise, produced by the system under study, including sensors noise;
2) Activity of different non-correlated sources, falling at the same frequency band

$${v}_{n} \pm \frac{1}{2 T} .$$

3) Activity of several coherent sources, shifted in phase, having exactly the same frequency vₙ and also falling at the same frequency band

$${v}_{n} \pm \frac{1}{2 T} .$$

Non-correlated noise can be reduced or removed completely through experimental design. The second reason is typical for usual methods of Fourier analysis, especially for methods with moving or fractional windows. The precise Fourier transform can solve the issue of activity of different non-correlated sources by either increasing the recording time T or by tuning of the frequency grid to an exact frequency. Tuning also can be applied to the analysis of a short time series, where it is impossible to calculate a detailed spectra.

In the third case, simultaneous activity of coherent sources with different spatial structures can indicate their functional connection. To divide different coherent processes from the restored multichannel signal the independent component analysis can be used or those processes can be split directly. This is described in greater detail below. This leads to the extraction of patterns, produced by several different sources with high coherence.

The algorithm of mass precise frequency-pattern analysis can be summarized as:

1. Precise Fourier Transform of the multichannel signal.

2. Inverse Fourier Transform-restoration of the signal at each frequency.

3. If the coherence is close to 1, then use the pattern and frequency as an elementary coherent oscillation.

4. If the restored signal consists of several phase-shifted coherent oscillations, then extract those oscillations.

After the fourth step, the initial multichannel signal will be represented as a sum of elementary coherent oscillations. Coherent oscillations are those oscillations of all or a majority of channels with a common phase, constant normalized patterns, and similar time course. For example, constant normalized patterns can be found from the signals even though the amplitude of a particular signal can vary with time. The normalized patterns are the same during the time of measurement. Coherent oscillations are produced by part, which is referred to as a functional entity, of the system under study. Each elementary oscillation can be characterized by an amplitude, a frequency, a phase, and a constant normalized pattern. In one embodiment, each elementary oscillation has a distinct frequency, a constant pattern and is produced by the functional entity having constant spatial structure. The initial multichannel signal can be represented as:

$${B}_{i} ( t ) = \frac{{a}_{0 i}}{2} + {\sum }_{n = 1}^{N} {\sum }_{m = 1}^{M} {a}_{mni} cos ( 2 π {v}_{n} t ) + {\sum }_{n = 1}^{N} {\sum }_{m = 1}^{M} {b}_{mni} sin ( 2 π {v}_{n} t ) .$$

The initial multichannel signal could also be written as:

$${B}_{i} ( t ) = \frac{{a}_{0 i}}{2} + {\sum }_{n = 1}^{N} {\sum }_{m = 1}^{M} {ρ}_{mni} sin ( 2 π {v}_{n} t + {ϕ}_{mn} )$$

$${v}_{n} = \frac{n}{T} , N = {v}_{max} T , m = 1 , … , M ,$$

where M is maximal number of coherent oscillations, extracted at the frequency vₙ. The number of elementary coherent oscillations can be large. Determining which elementary coherent oscillations are useful can be time consuming. Below are various ways that can be used to identify which elementary coherent oscillations to further analyze.

The above decomposition provides a set of patterns. To classify and to describe them systematically, the projection to an orthogonal system of functions can be used. For example, the following formula can be used:

$${ρ}_{n} ( i ) = {\sum }_{k = 1}^{K} {a}_{k} {f}_{k} ( i ) ,$$

where ρₙ (i) describes a coherent pattern at the frequency vₙ, {ƒₖ(i)} is the set of Karhunen-Loeve eigenfunctions, or any other orthogonal basis in channels space, and αₖ is the pattern projections to K-L space. These projections provide an objective quantitative description of the pattern, making it possible to classify and to compare patterns. The projection can be applied to each pattern, so at the particular frequency there can be several decompositions. Differences between projected patterns can be calculated as:

$${E}_{proj} = {\sum }_{k = 1}^{K} {{w}_{k} ( {a}_{k}^{( {n}_{1} )} - {a}_{k}^{( {n}_{2} )} )}^{2} ,$$

where wₖ are weights of the K-L functions. Those weights can be used, for example, to eliminate noise or to emphasize definite features of the patterns. Other methods of classification can be also applied to projected patterns.

In another implementation similar patterns can be determined as follows. For every frequency, the normalized pattern can be calculated as:

$${\hat{ρ}}_{n} ( i ) = \frac{{ρ}_{n} ( i )}{{D}_{n}} ,$$

where ρₙ (i) describes a coherent pattern and

$${D}_{n} = \sqrt{{\sum }_{i = 1}^{K} {ρ}_{n}^{2} ( i )} ,$$

which is a square root of the summary energy for this pattern. This operation provides the normalized pattern as:

$${\sum }_{i = 1}^{K} {\hat{ρ}}_{n}^{2} ( i ) = 1 ,$$

that describes spatial features of the image apart from its intensity. Differences between two normalized patterns \hat{ρ}ₙ_{₁}(i) and \hat{ρ}ₙ_{₂}(i) can be calculated directly:

$$E = \frac{1}{2} {\sum }_{i = 1}^{K} {( {\hat{ρ}}_{{n}_{1}} ( i ) - {\hat{ρ}}_{{n}_{2}} ( i ) )}^{2} .$$

For any pair of patterns 0≦E≦1. Two patterns can be called similar with precision ε, if E<ε. For two identical patterns E=0, for two orthogonal patterns E=1

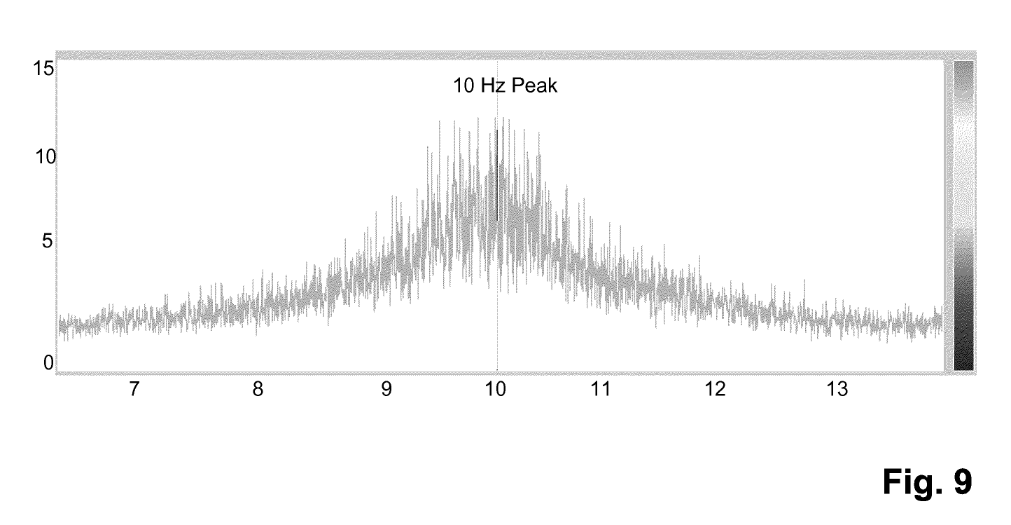

As one example, a pattern can be selected from a spectrum. For example, the peak from FIG. 9 can be selected as a pattern. The peak can be decomposed to its independent coherent components, providing two field patterns. Each of these patterns can be normalized and used as a trial pattern. This allows further study of the selected peak.

Using the normalized patterns described above, the total decomposition of the system into elementary coherent oscillations of the functionally invariant entities can be written as:

$${B}_{i} ( t ) = {D}_{10} {\hat{ρ}}_{10 i} + {\sum }_{n = 1}^{N} {\sum }_{m = 1}^{M} {D}_{mn} {\hat{ρ}}_{mni} sin ( 2 π {v}_{n} t + {ϕ}_{mn} )$$

Each elementary oscillation is characterized by frequency vₙ, phase φₘₙ, amplitude Dₘₙ, and normalized pattern \hat{ρ}ₘₙᵢ.

The partial spectrum can be assembled by the following algorithm:

1. Build or select a trial normalized pattern \hat{ρ}ᵢ^{tr}, i=1, . . . , K.

2. Estimate the difference between the trial pattern and every pattern of elementary oscillation, using the projections αₖ as a features or directly:

$${E}_{mn} = \frac{1}{2} {\sum }_{i = 1}^{K} {( {\hat{ρ}}_{i}^{tr} - {\hat{ρ}}_{mni} )}^{2}$$

3. Specify the accuracy of patterns comparison ε.

4. Specify the filtering function ƒ(Eₘₙ). The exemplary (but not limited) filtering function can be determined as:

if Eₘₙ<ε, than ƒ(Eₘₙ)=1, else ƒ(Eₘₙ)=0.

5. Write the partial spectrum, corresponding to the trial pattern ρᵢ^{tr}, as:

$${B}_{i} ( t ) = f ( {E}_{10} ) {D}_{10} {\hat{ρ}}_{10 i} + {\sum }_{n = 1}^{N} {\sum }_{m = 1}^{M} f ( {E}_{mn} ) {D}_{mn} {\hat{ρ}}_{mni} sin ( 2 π {v}_{n} t + {ϕ}_{mn} ) .$$

This formula reconstructs the time dependence of the functional entity, oscillating as a whole with accuracy ε. Spatial structure of this functional entity can be estimated from the trial pattern ρᵢ^{tr}.

The partial spectrum can be utilized in various applications. For example, if the functional entity, providing this spectrum, is identified as the object of current study, then conclusions can be made about this object. For example, the location or the path through a body as a result of the object can be determined. If that functional entity is identified as an interference generator, then its partial spectrum can be subtracted from the general spectrum, leading to further cleaning of the data.

Based upon the filtered functions, the sum of the energy, produced by the functional entity, corresponding to the trial pattern \hat{ρ}ᵢ^{tr} can be characterized by

$${E}^{tr} = {f}^{2} ( {E}_{10} ) {D}_{10}^{2} + {\sum }_{n = 1}^{N} {\sum }_{m = 1}^{M} {f}^{2} ( {E}_{mn} ) {D}_{mn}^{2} .$$

Using the representative set of the trial patterns, it is possible to calculate corresponding energies. The distribution of those energies can provide information about the functional structure of the system. This is illustrated in the “Analysis of the MEG Partial Spectra for Thalamic Brain Activity” and “Functional Tomography of the Human Brain Based on Magnetoencephalography” examples below.

After identifying the partial spectrum associated with a trial pattern, a functional tomogram can be produced. A functional tomogram can display a 3-dimensional map of the energy, produced by all sources, located at a given point in space. In order to build a functional tomogram of a given space, the space is divided into Nₓ×N_{y}×N_{z} elementary blocks, e.g., voxels. The energy contained within each of the elementary blocks produced by all sources can be calculated and assigned to the center of the block. In other implementation, a 3-dimensional vector field of the dominant directions of the sources can be plotted on the functional tomogram.

After selecting the set of frequencies to study, the inverse problem for every pattern ρₘₙᵢ is solved. This solution to the inverse problem for a particular pattern describes the spatial structure of the entity that is producing the particular pattern. The dipole moment or the current density found from this solution is associated with the nearest elementary block. The sum of contributions from all solutions of inverse problem for elementary block is calculated, and the result is assigned to its center. The 3D-array obtained represents the functional tomogram, based on solutions of inverse problem for the patterns of elementary coherent oscillations. An example of a functional tomogram is described in the “Functional Tomography of the Human Brain Based on Magnetoencephalography” below.

### Estimation of Spatial Structures and Restoration of Time Courses of the Functional Entities.

As follows from the Coherence Theorems above, each elementary oscillation, described in terms frequency-pattern, corresponds to a distinct functional entity. It also follows, that it is possible to restore the time course of the particular entity, selecting all elementary oscillations produced by this entity and having the similar patterns.

This selection can be done using the projections αₖ as a features. After the description and classification of coherent patterns, the inverse problem for each pattern selected can be solved. This gives the decomposition of the system under study to the set of functional entities, producing the whole activity measured in experiment. As follows from the Coherence Theorem 3, the time course of the particular signal can be restored by the Inverse Fourier Transform performed over the spectra, combined from all frequencies with similar patterns.

The above describes determining a time course of a signal and the spatial-structure of components in a body, which can be used in various different ways. The sensors used to collect the data can be various types of sensors. The following non-limiting examples describe some of the different ways a signal and the spatial-structure of components in a body can be determined.

### Magnetic Cardiogram Analysis

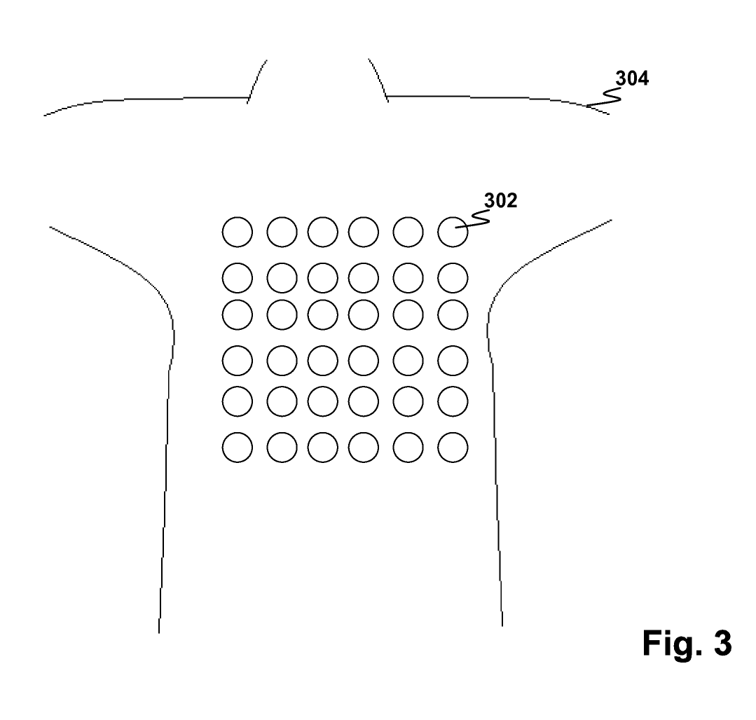

Heart activity can be monitored using multiple sensors and analyzing the recorded data as described above. The heart as well as other monitored components can generate spontaneous signals that are not in response to an external stimulus. These signals can be recorded and analyzed. As an example, in one experiment 9 separate monitors, each corresponding to a channel, were used to measure heart magnetic activity. In the experiment, the sensors were 2^{nd} order gradiometers that were highly sensitive SQUID-based magnetic field detectors immersed in a liquid helium filed Dewar. The sensors were distributed equidistantly on a square surface and placed over a subject's chest in 4 positions, giving 36 channels. FIG. 3 illustrates spacing of the sensors in the heart monitoring experiment. Thirty-six sensors, e.g., 302, were placed in a square over the chest of a subject 304.

All 36=9×4 channels of the magnetic cardiogram were utilized. In addition, for every position of the device one channel of the electrocardiography was simultaneously utilized. The channel of the electrocardiography was used to compare with the MCG data. The sampling frequency was 1000 Hz, and the registration time was 30 seconds. Each sensor measured data independently, affording dynamic information regarding magnetic field at each sensor position.

### Precise Filtering of the Magnetic Cardiogram

The experiment was performed without a magnetically insulated room, resulting in a high level of noise. Useful signals, however, can be analyzed even in light of the noise. Using the precise Fourier transform as described above, resulted in multichannel spectra covering the whole recording time period (30 seconds, giving a frequency resolution of 1/30 Hz).

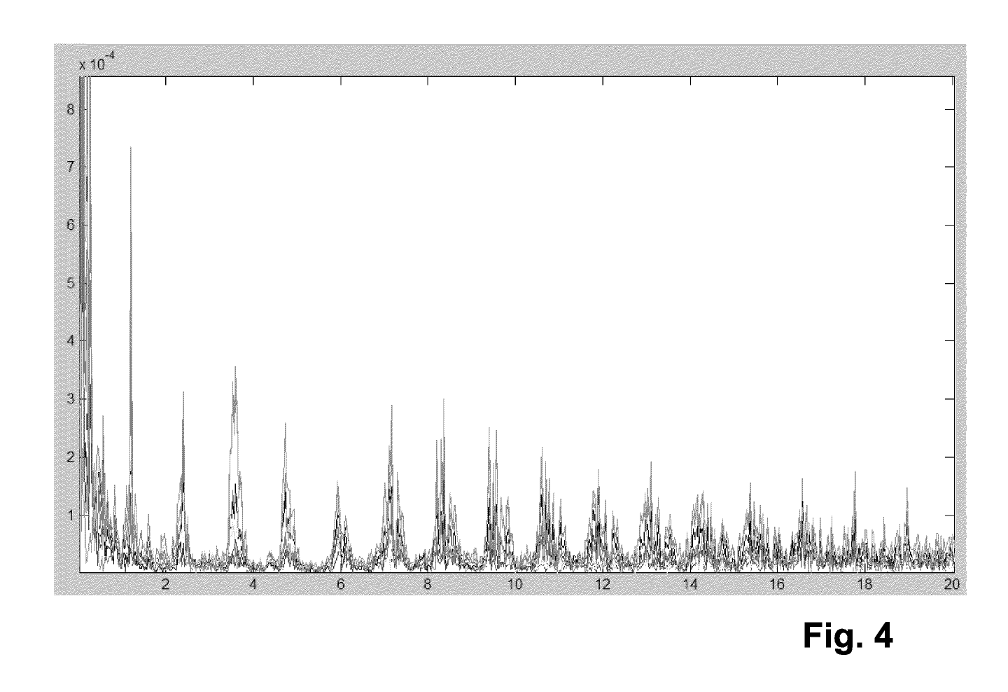

FIG. 4 is magnetic cardiogram multichannel spectrum in accordance with an illustrative embodiment. As can be seen in FIG. 4, the spectra separate into well-defined frequency peaks, many of them with high coherence profiles affording constant patterns of the magnetic field. These patterns are sufficiently robust to support unambiguous source localization. It is also clear, that the MCG spectra contain a great number of peaks corresponding to the harmonic of basic heartbeat frequency (about 1.2 Hz). Accordingly, the precise frequency-pattern analysis can be used to classify the sources of the heart activity at the basic heartbeat frequency, e.g., 1.2-1.3 Hz.

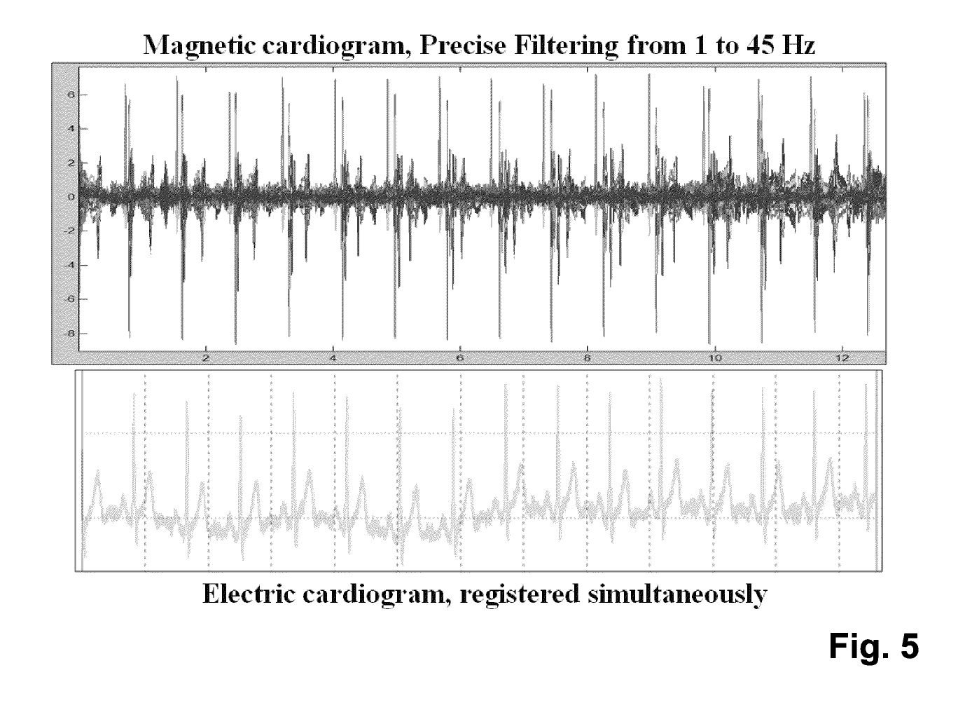

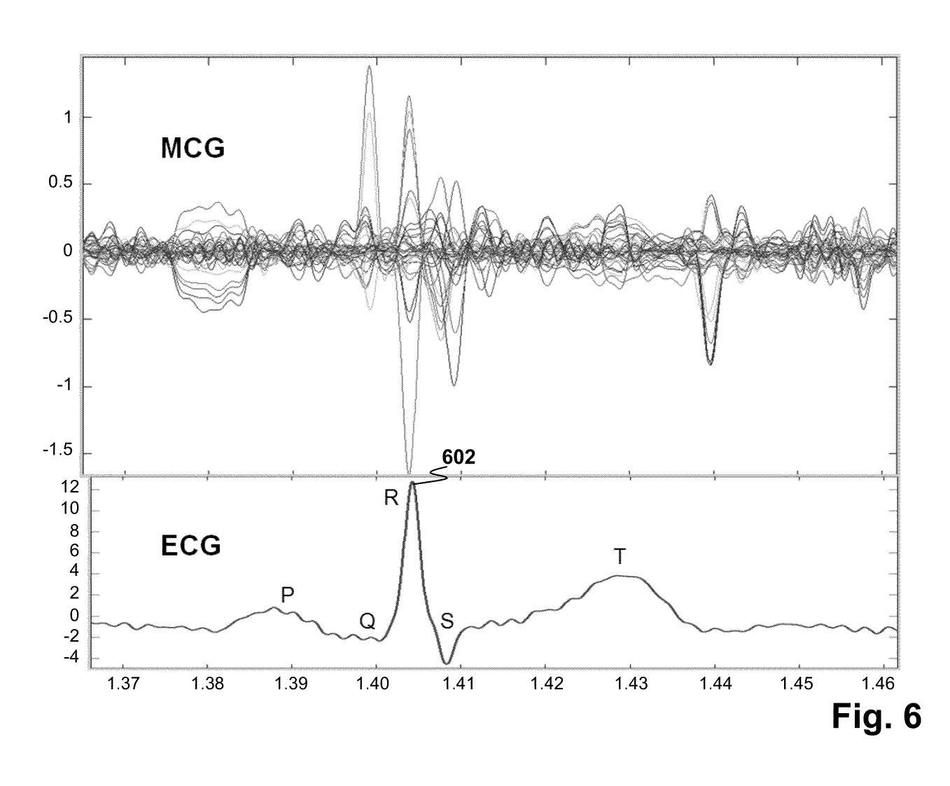

Using the inverse Fourier Transform described above the general time course can be restored. FIGS. 5 and 6 illustrate a comparison between MCG and ECG data in accordance with an illustrative embodiment. FIG. 5 illustrates the restored time course and shows that all the magnetic heartbeat cycles (throughout the whole recording time) were restored. The heartbeat cycles from the MCG data were in correlation with the simultaneously recorded electrocardiogram as shown in both FIGS. 5 and 6. The MCG recordings therefore, allow the possibility to study activity of the heart in details, including pattern analysis and inverse problem solution for any moment of time.

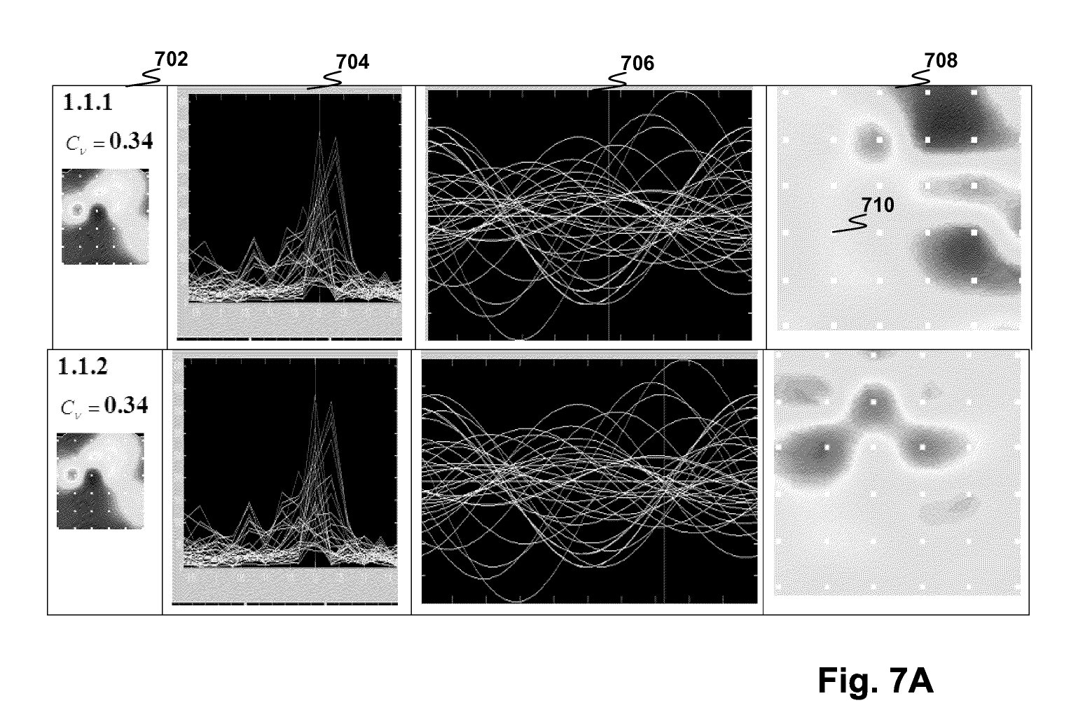

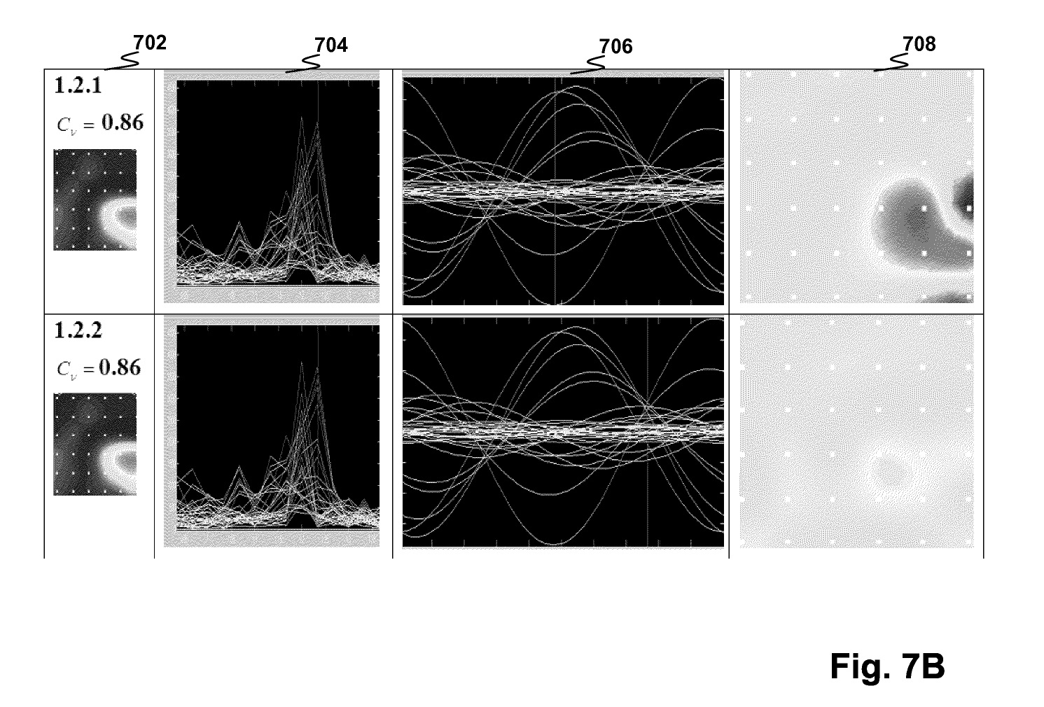

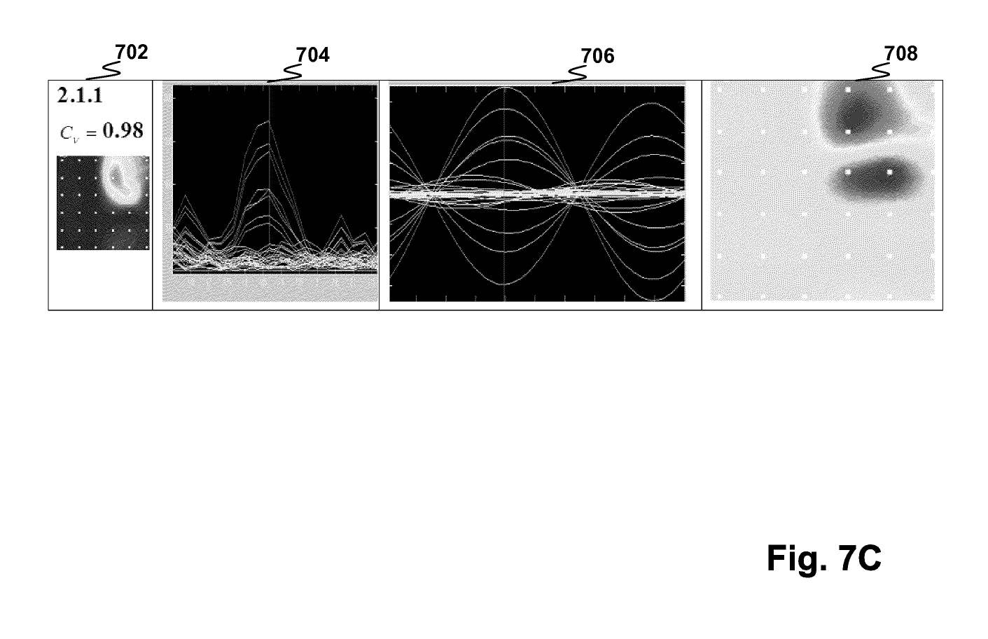

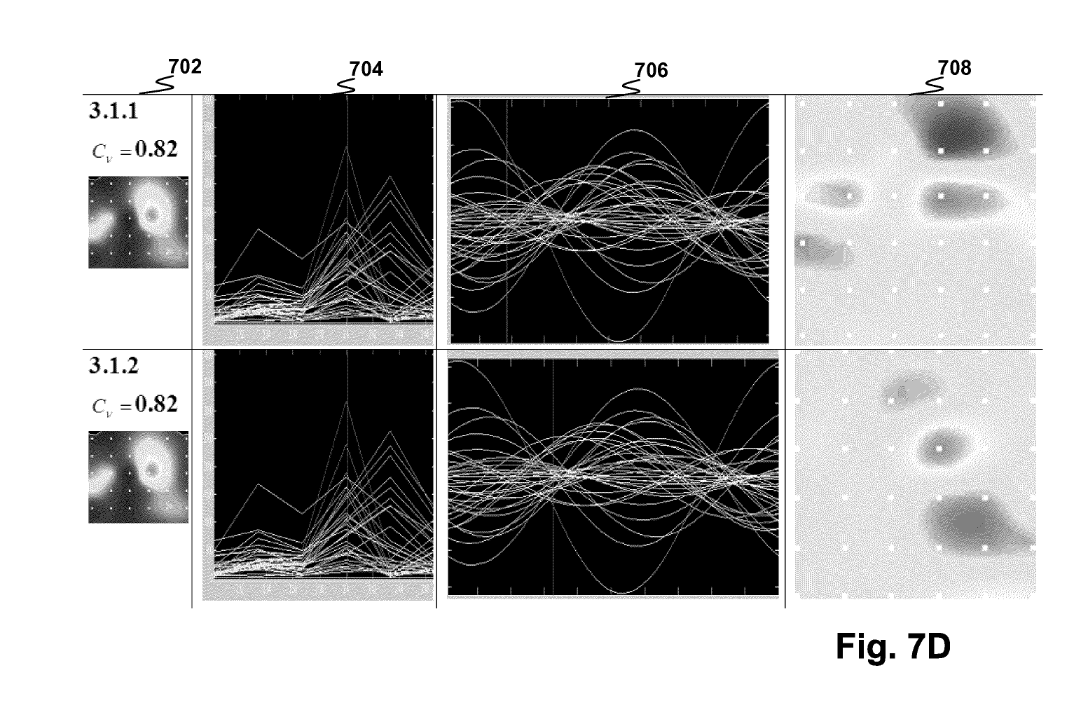

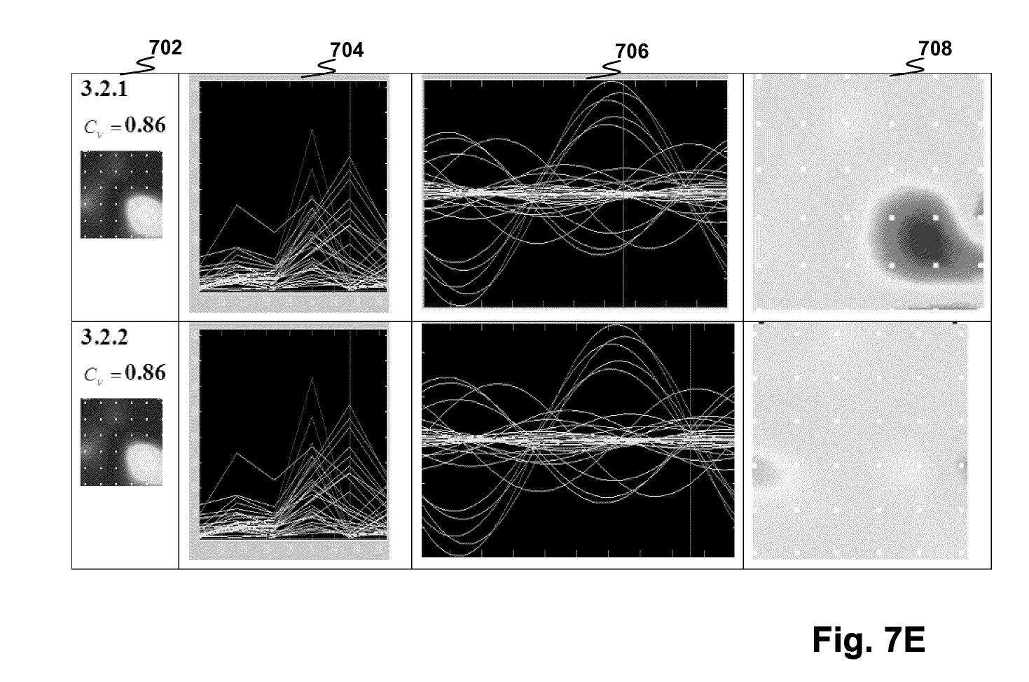

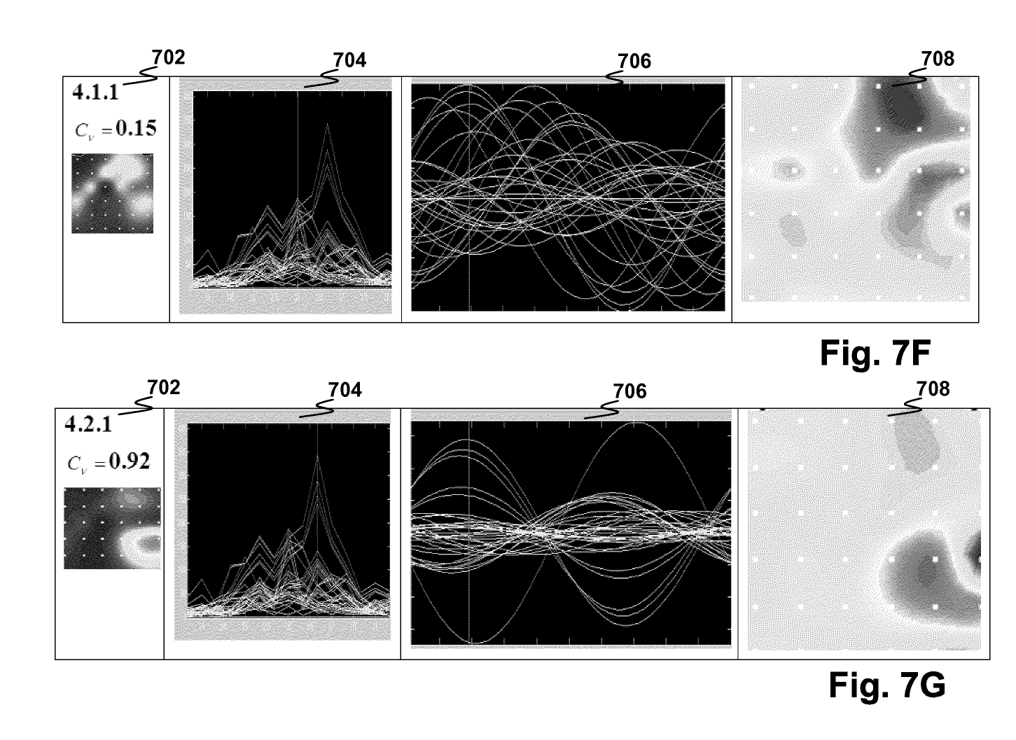

FIGS. 7A-7G illustrate the precise frequency-patter analysis and coherence analysis for MCG data in according with an illustrative embodiment. These figures show the coherence value Cᵥ for a group of peaks 702; the precise multichannel spectrum for the frequency band near a harmonic 704; time courses of the reconstructed signals in all channels for the period of the harmonic 706; and the magnetic field patterns at the moment of maximal instantaneous power 708. The various shades of the magnetic field patterns indicate the direction of the field. Regions ringed in white indicate that the direction of the field is up; darker regions indicate that the direction of the field is down. Intensity of the shading corresponds to the amplitude of the induction, with darker regions corresponding to larger amplitudes. The sensors 710, as white squares, are also shown in the magnetic field patterns.

FIG. 7A illustrate a complex spectra at a basic frequency corresponding to a first peak near the heartbeat frequency. The first peak reveals low coherence, because is contains two oscillations, e.g., FIG. 7A, with different patterns at the same frequency. It is possible that this shows the cooperative functioning of two sources. FIG. 7B illustrates data corresponding to a second peak near the heartbeat frequency. This data also contains two oscillations, although the peak is more coherent and better localized. FIG. 7C shows a wide peak with an extremely high coherence. The oscillation illustrated in FIG. 7C reveals the source and pattern corresponding to the pattern of an R-peak 602 in the ECG portion of FIG. 6. The R-peak 602 is the most energetic peak in the ECG data of FIG. 6. FIG. 7D illustrates data associated with the second harmonic of the heartbeat, which is also complex. FIG. 7E, similar to FIG. 7B, shows the basic frequency is complex. FIGS. 7F and 7G illustrate data associated with the heartbeat's third harmonic. The third harmonic is also complex. The first peak of the third harmonic, illustrated in FIG. 7G, has a low coherence. The second peak of the third harmonic, illustrated in FIG. 7G, however, has a high coherence.

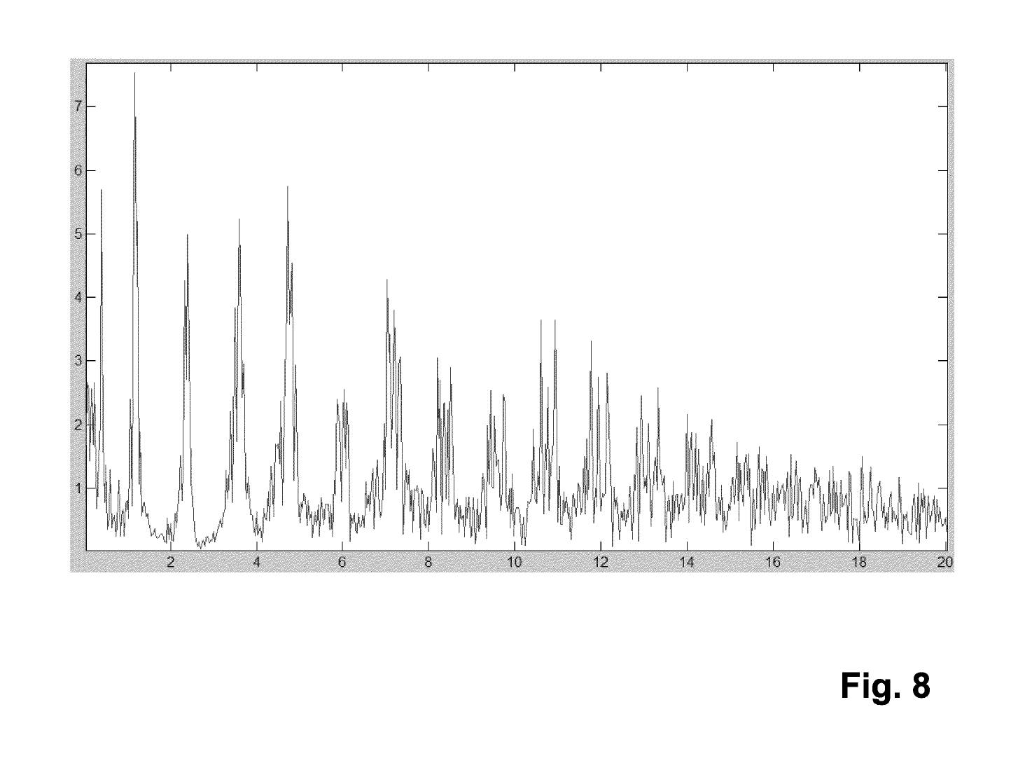

In addition to MCG data, the precise Fourier transform described above can be applied to ECG data. For example, the precise Fourier transform was applied to the one-channel recording of the ECG signal. The ECG spectrum, shown in FIG. 8, has a structure similar to MEG and MCG spectra. Using the coherence theorems above, the precise frequency-pattern analysis can be applied to multichannel ECG data.

### Alpha-Rhythm Analysis in Magnetic Encephalography

Precise frequency-pattern analysis can also be used to analyze multiple components of alpha-rhythm spontaneous activity. This activity can be split to coherent components, as shown below. FIG. 9 is a graph of an alpha-rhythm peak in the general spectrum and shows a peak near 10 Hz. In an experiment, multiple sensors, e.g., 275 sensors, can equidistantly cover the whole surface of a subject's head. The sensors can be highly sensitive SQUID-based detectors of a magnetic field and can be immersed into a Dewar with liquid helium. The sensors can then be placed over a helmet at the bottom of the Dewar.

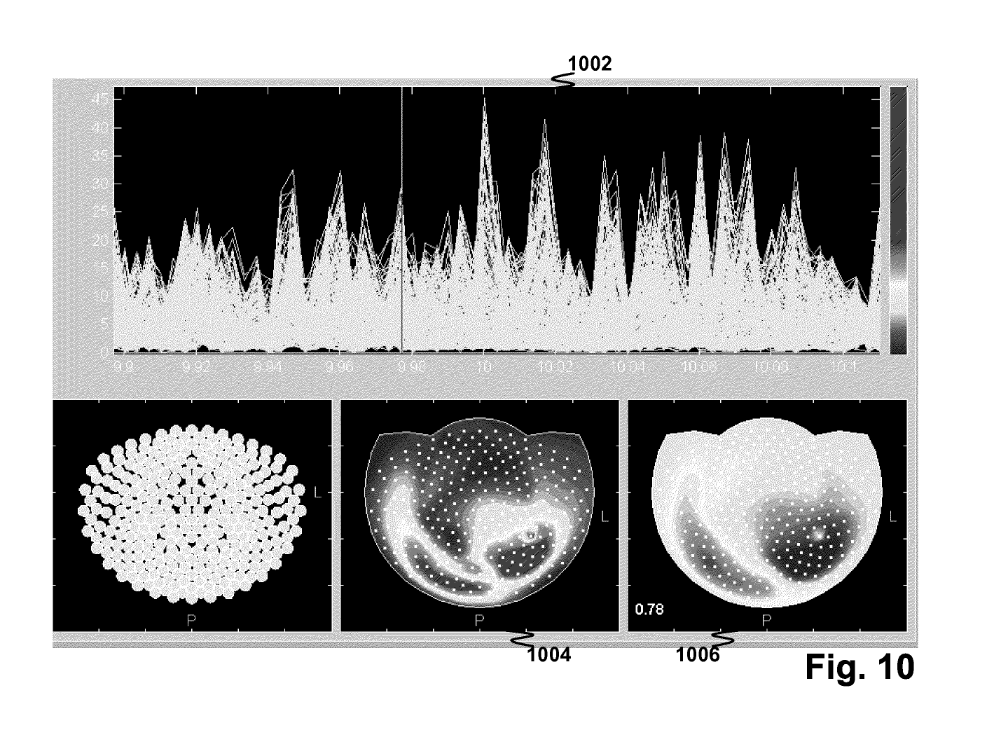

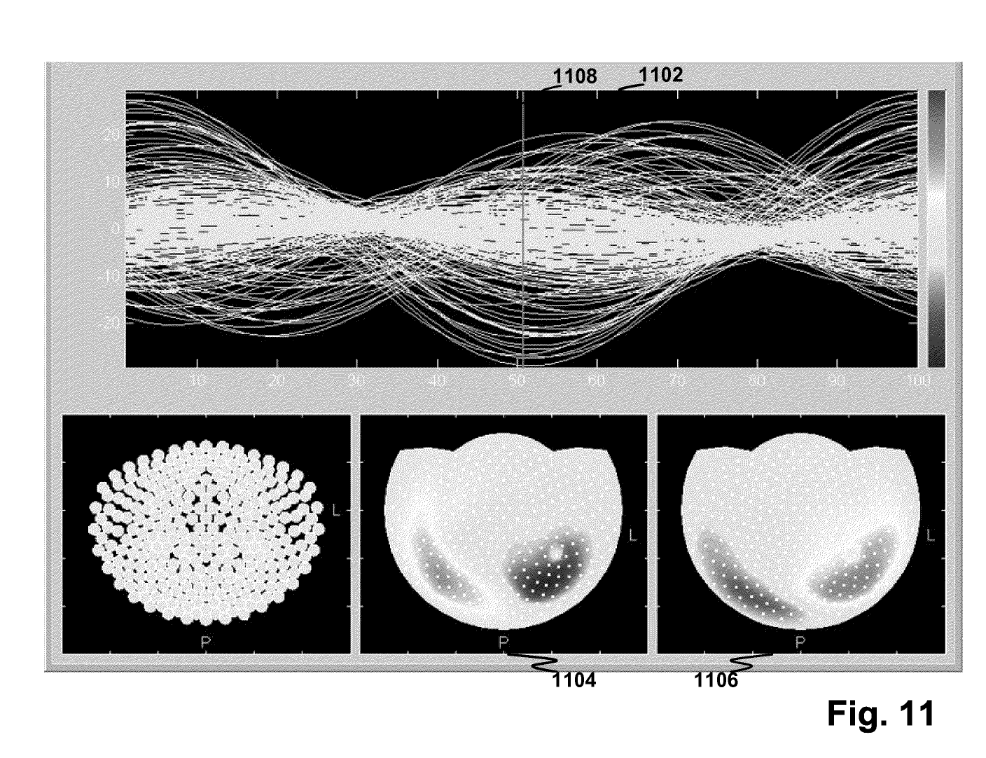

FIGS. 10-12 illustrate a user interface to analyze alpha-rhythm peaks in accordance with an illustrative embodiment. A multichannel spectrum window 1002 shows the precise multichannel spectrum for a frequency range, e.g., results of the precise Fourier transform of the data from each of the sensors. The spectrum range in the illustrated multichannel spectrum window 1002 covers the 10 Hz peak from FIG. 9. The spectra pattern is shown in window 1004 and the field pattern is shown in window 1006. Different shading represent different amplitudes in the window 1004 of the spectrum and different directions of the field in the window 1006. A coherence score related to the field pattern can also be displayed in the window 1006.

FIG. 11 shows a window for the analysis of the restored multichannel signal, e.g., results of the inverse Fourier transform into the time-domain. Window 1102 shows the restored signal for each of the channels. A cursor 1108 can be used to select the moment in time to use for extraction of the source pattern. Windows 1104 and 1106 show the field pattern. The window 1104 shows the experimental or restored field and the window 1106 shows the inverse problem solution for this or a previous pattern. Different shading represents different directions of the fields in the window 1106. As the cursor 1108 is moved to different times, the data illustrated in windows 1104 changes accordingly.

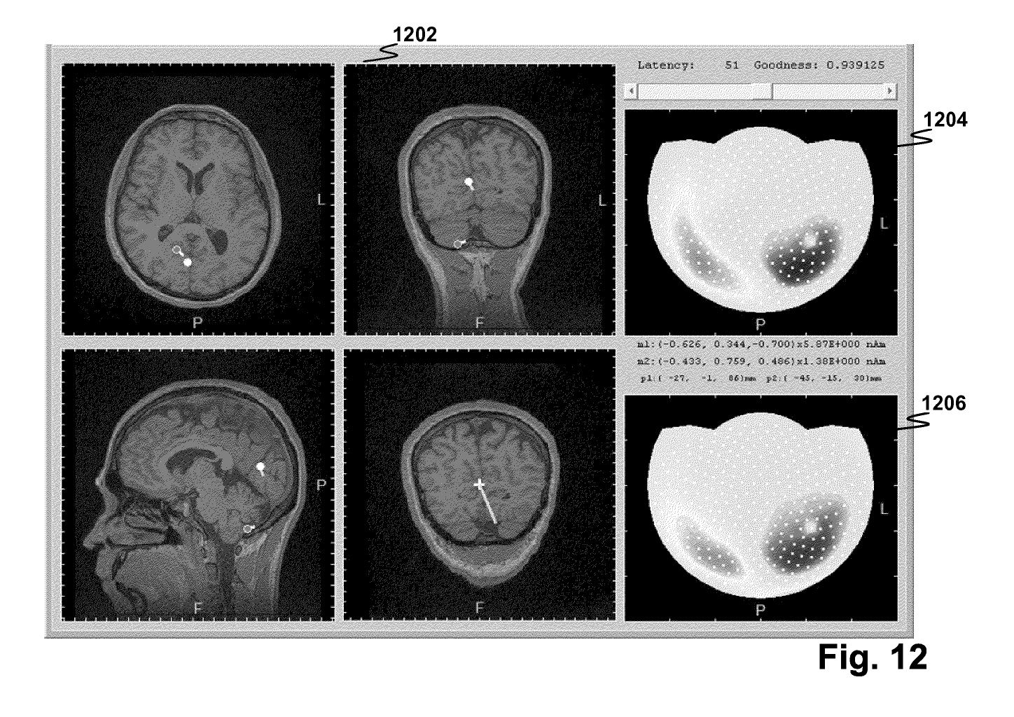

FIG. 12 illustrates a window for the inverse problem solution. Magnetic resonance images 1202 show the subject under study. The magnetic resonance images 1202 show the position of two dipoles. The position of the two dipoles is found by the inverse problem solution. Window 1204 shows the pattern of the experimental or restored field. Window 1206 shows the pattern of the field found by the inverse problem solution.

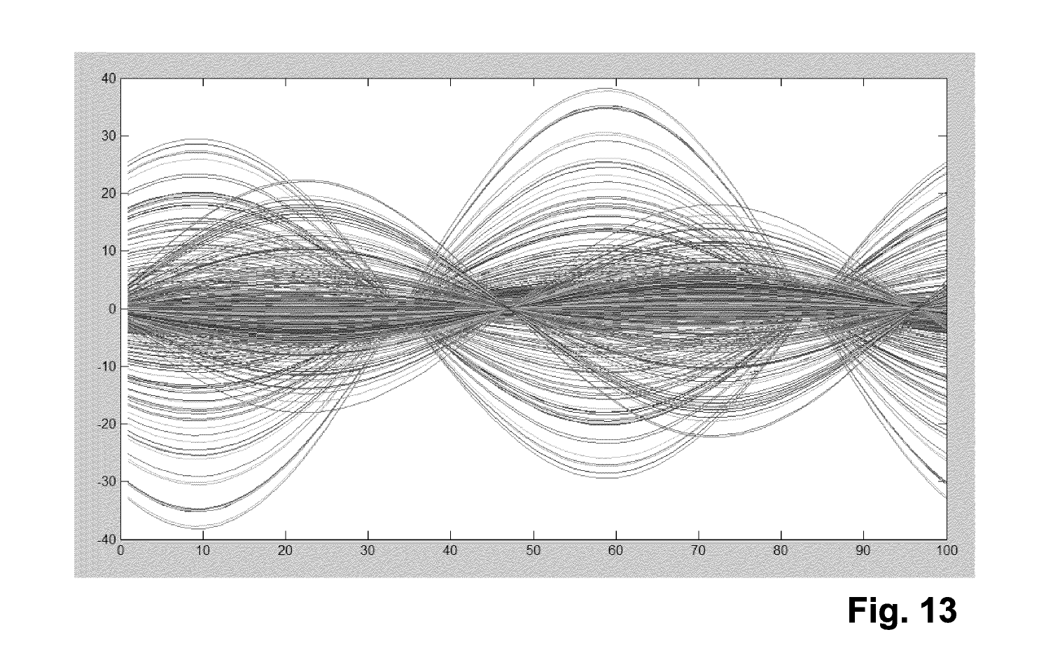

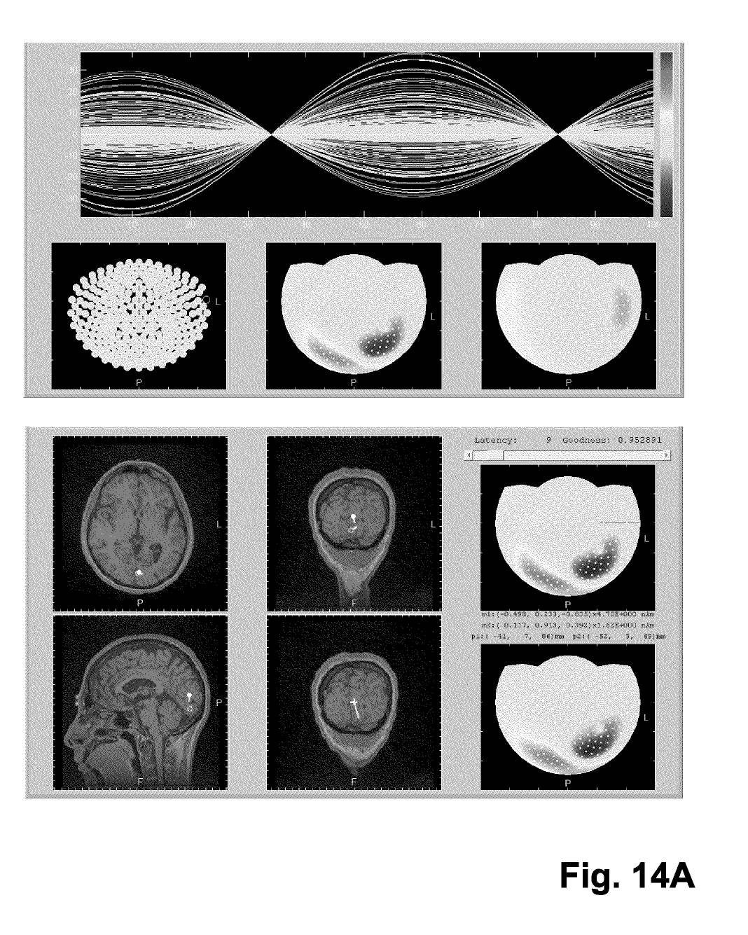

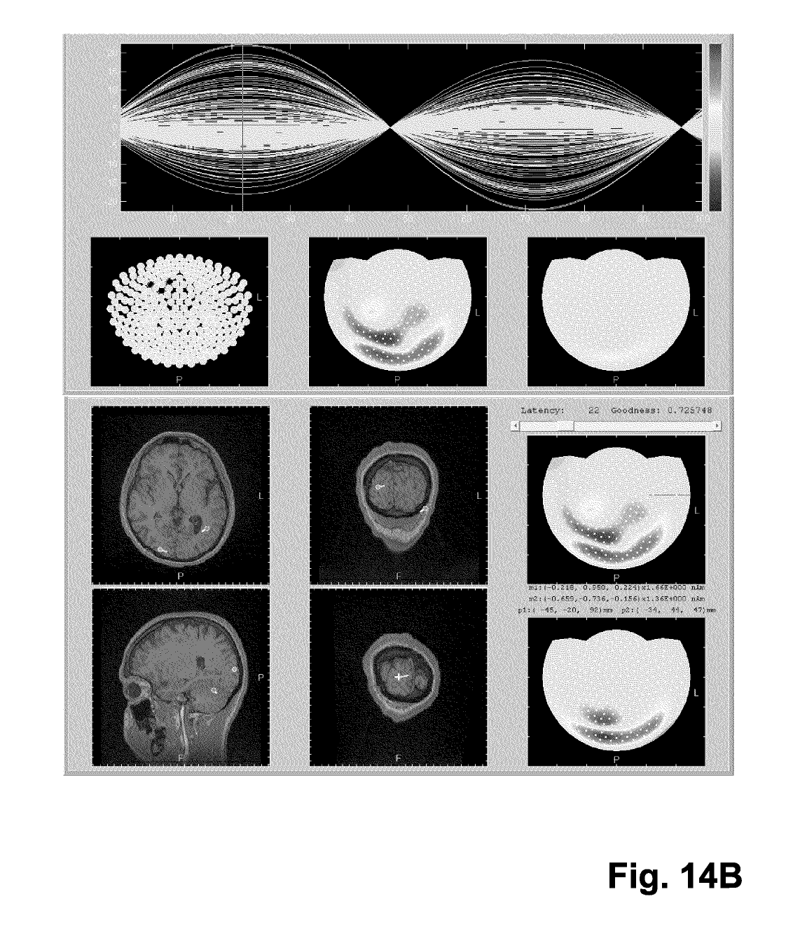

Independent component analysis can be used to identify several coherent oscillations with exactly the same frequency that are phase shifted with respect to one another. FIG. 13 illustrates two oscillations that are phase shifted. As described above, activity of several coherent sources with different spatial structures at the same frequency can indicate their functional connection. In one example, the independent component analysis was applied to the restored signal and revealed two coherent signals, as illustrated in FIG. 13. In FIG. 13, the phase shift is roughly 13 milliseconds. The inverse problem solution for each of the two coherent signals can be determined. FIGS. 14A and 14B illustrate the inverse problem solution for each of the two coherent signals in accordance with an illustrative embodiment. Comparing the location of the dipoles in FIGS. 14A and 14B, the dipoles have a different direction and are located in different portions of the subject's brain. Accordingly, the two coherent signals are part of the same process, occurring in the illustrated positions in space.

### Precise Frequency-Pattern Analysis of the Magnetic Encephalography Data

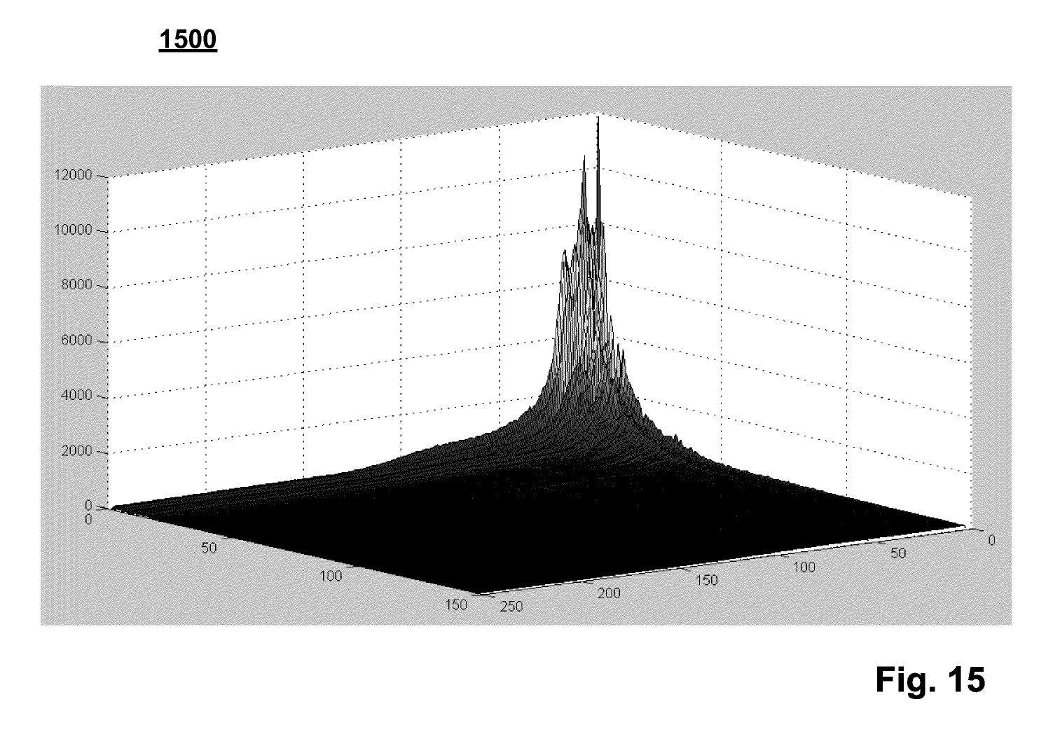

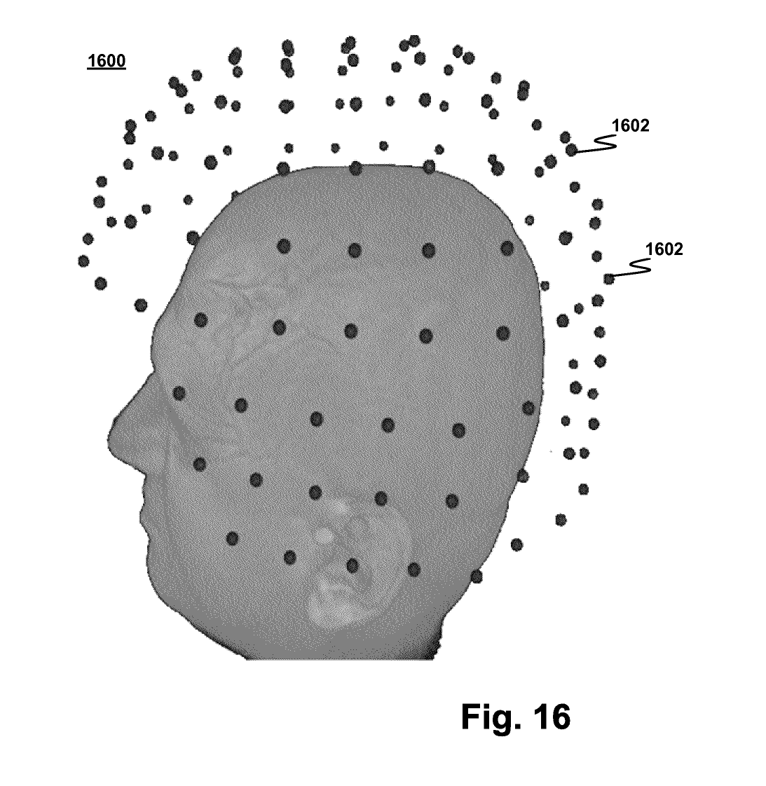

To increase the resolution of the MEG data in regard to localization of either spontaneous and/or evoked magnetic fields, a precise Fourier transform can be used. FIG. 15 illustrates a MEG 3-dimensional spectrum 1500 in accordance with an illustrative implementation. As seen from the spectrum 1500, signal energy is limited in frequency and space. MEG data can be collected from a number of devices, such as, but not limited to, e.g., magnetometers, gradiometers, etc., that are arranged around the head of a subject. FIG. 16 illustrates devices used to measure magnetic fields of a subject in accordance with an illustrative implementation. Devices 1602 can be arranged around a subject's head in various known ways. For example, in one implementation 148 devices can be used to measure the magnetic fields of the subject. As described above, two different types of responses can be analyzed based upon the data collected from the devices 1602. Spontaneous activity is activity that occurs without an external stimulus. Evoked activity is activity that occurs based on an external stimulus. For example, an evoked activity can be based upon an audio tone at a particular frequency.

For spontaneous activity analysis, a precise Fourier transform of the data from all of channels can be done. In one implementation, the following mathematical expression is used for the Fourier series:

$$f ( t ) = \frac{{a}_{0}}{2} + {\sum }_{n = 1}^{N} {a}_{n} cos ( 2 π {v}_{n} t ) + {\sum }_{n = 1}^{N} {b}_{n} sin ( 2 π {v}_{n} t ) , \\ {v}_{n} = \frac{n}{T} , N = {v}_{max} T$$

$${a}_{0} = \frac{2}{T} {{\int }_{0}}^{T} f ( t ) d t , {a}_{n} = \frac{2}{T} {{\int }_{0}}^{T} f ( t ) cos ( 2 π {v}_{n} t ) d t , \\ {b}_{n} = \frac{2}{T} {{\int }_{0}}^{T} f ( t ) sin ( 2 π {v}_{n} t ) d t ,$$

where T is the time of registration, a₀, aₙ, bₙ are Fourier coefficients for the frequency vₙ, N is the maximal number of frequencies, and vₘₐₓ is the highest desirable frequency. The data from all of the channels is interpolated to a continuous function, e.g., ƒ(t). Then Gaussian quadratures are used to calculate integrals on any interval [0,T] in the scale of registration.

Using the above mathematical expressions, the step in frequency is calculated as:

$$Δ v = {v}_{n} - {v}_{n - 1} = \frac{1}{T} .$$

Frequency resolution, therefore, is determined by the time of registration, so the T must be sufficient to reveal detailed frequency structure of the system, even in studies using moving or fractional window. The above described transform generates accurate and reversible presentation of time data in the frequency domain for every channel. Spatial data can be determined by collecting data from many different channels.

After the precise Fourier transform of the data from all of the channels, an estimate of the multichannel spectrum and selection of frequencies of interest can be done. Based upon the selected frequencies, an inverse transform can be done for the selected frequencies in all of the channels. Frequencies corresponding to noise can be ignored. Identification of noise signals is described in greater detail below. The inverse transform of frequencies allows for the localization of a source from the data. That is, activity occurring within the brain which corresponds to the selected frequencies can be localized. In one implementation, localization can be done as follows. For a selected moment of time, the root-mean-square deviation between model and experimental values of the magnetic induction can be minimized.

Σₖ₌₁^{K}(Bₖ−Bₖ⁰)²→min.

Here Bₖ⁰= m̅(tᵢ,k) is the magnetic induction measured by the k-th sensor at the moment t, while Bₖ=(B(rₖ),nₖ) is the induction of the model field calculated for the position of the k-th sensor rₖ and its direction nₖ. When evoked activity is to be measured, the sources of auditory activity can be simulated by two equivalent current dipoles. Induction B(rₖ) can be calculated as a sum of two magnetic dipoles, each being described by the model of a current dipole in a conducting sphere:

$$B ( r ) = - {μ}_{0} ∇ U ( r ) , U ( r ) = - \frac{1}{4 π} \frac{( Q \times {r}_{0} , r )}{F}$$

$$F = a ( ar + {r}^{2} - ( {r}_{0} , r ) ) , a = r - {r}_{0} , {μ}_{0} = 4 π \cdot {10}^{- 7} .$$

Minimization with respect to the dipole position can be carried out by a Nelder-Mead simplex method. Minimization with respect to the dipole moment at a fixed position of the dipole can be done by solving the system of linear equations, since the magnetic induction depends linearly on the dipole moment. So, each trial calculation of the functional depended on two vector parameters, i.e., positions of two dipoles.

Further, as part of the selection of frequencies, a frequency grid can be used. The frequency grid is detailed enough in the frequency realm to be able to show spikes in a vary narrow frequency band. This allows for different peaks to be analyzed and/or ignored. The frequency grid can be tuned by cutting of the time of registration T, to build an optimal approximation of the frequency selected.

As the collection of data from the various channels occurs over a period of time, an estimation of the resulting multichannel time series of the data can be determined. That is, the source location corresponding to a frequency can be traced within the brain over the observed period of time.

In one experiment an evoked response can be analyzed from collected MEG data. In one example setup, experimental data was collected using a 148-channel magnetometer Magnes 2500 WH in the Bellevue Hospital in the Center for Neuromagnetism of New York University School of Medicine. Sensors were highly sensitive SQUID-based detectors of a magnetic field immersed into a Dewar with liquid helium. The coils' diameter was 2.3 cm, and the average distance between the centers of the coils approximated 2.9 cm. The sensors covered equidistantly the whole surface of the head and were placed over the helmet at the bottom of the Dewar. The sampling frequency was 500 Hz, and the time of registration was 300 seconds. Every sensor measured one channel of the data independently of other sensors, giving dynamic information about the magnetic field in its position. The data from each sensor can be collected and analyzed.

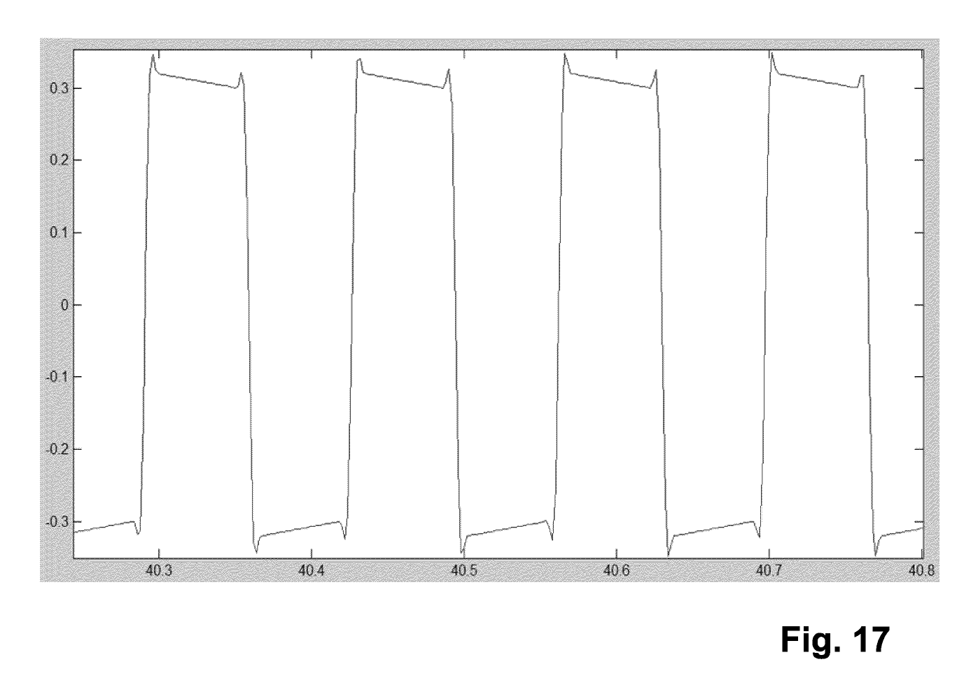

In the performed experiment, an external auditory stimulus was provided to the subject. FIG. 17 is a graph of a stimulus profile used in the MEG experiment in accordance with an illustrative implementation. The auditory stimulus was input at a frequency of 7.3821 Hz into the left ear of a healthy subject. The stimulus profile, illustrated in FIG. 17, was a plateau of a half-period length with rather sharp fronts. Time of measuring was T=300 seconds, containing 2,250 periods of the stimulus. As described above, data can be collected from a number of sensors that measure a magnetic field at a particular location relative to the subject. The idea of using the non-harmonic stimulus is to activate the auditory system of the brain on different frequencies, which are the harmonics of basic frequency.

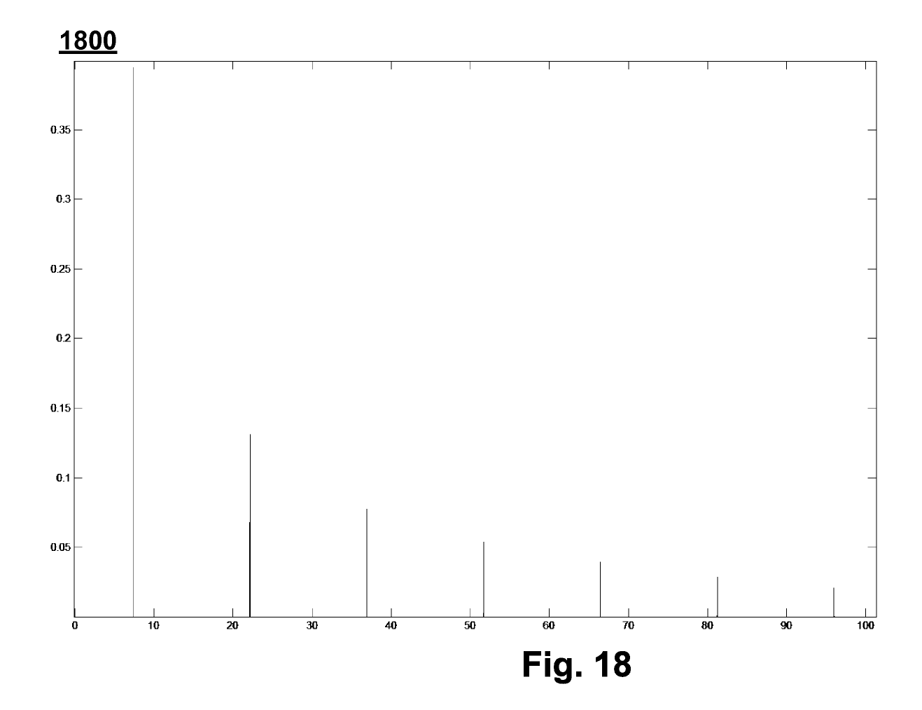

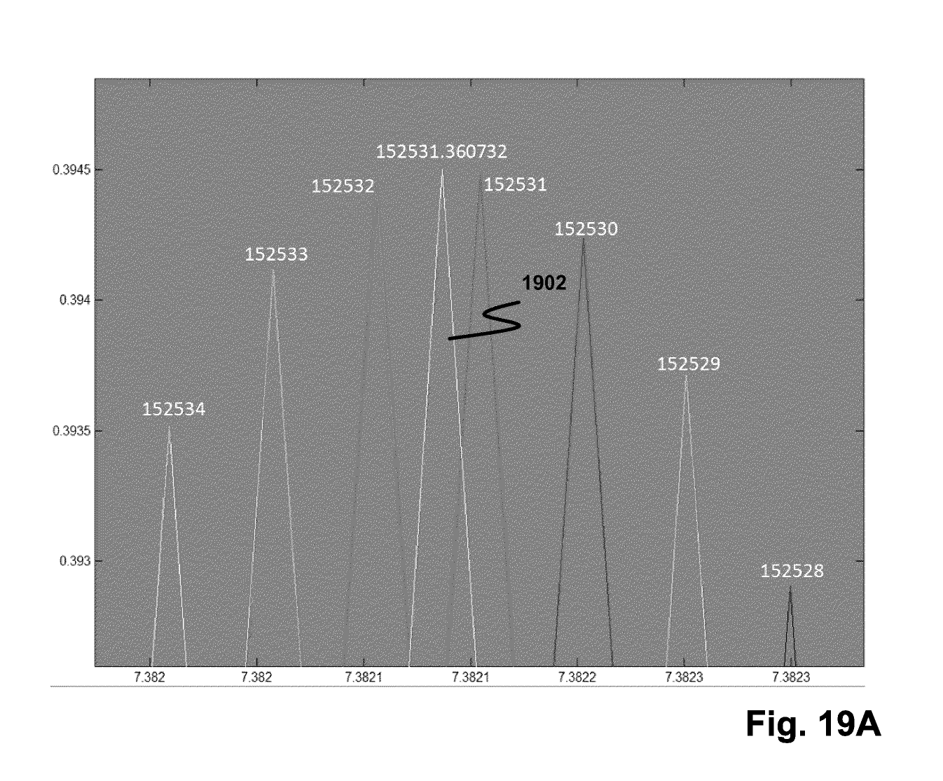

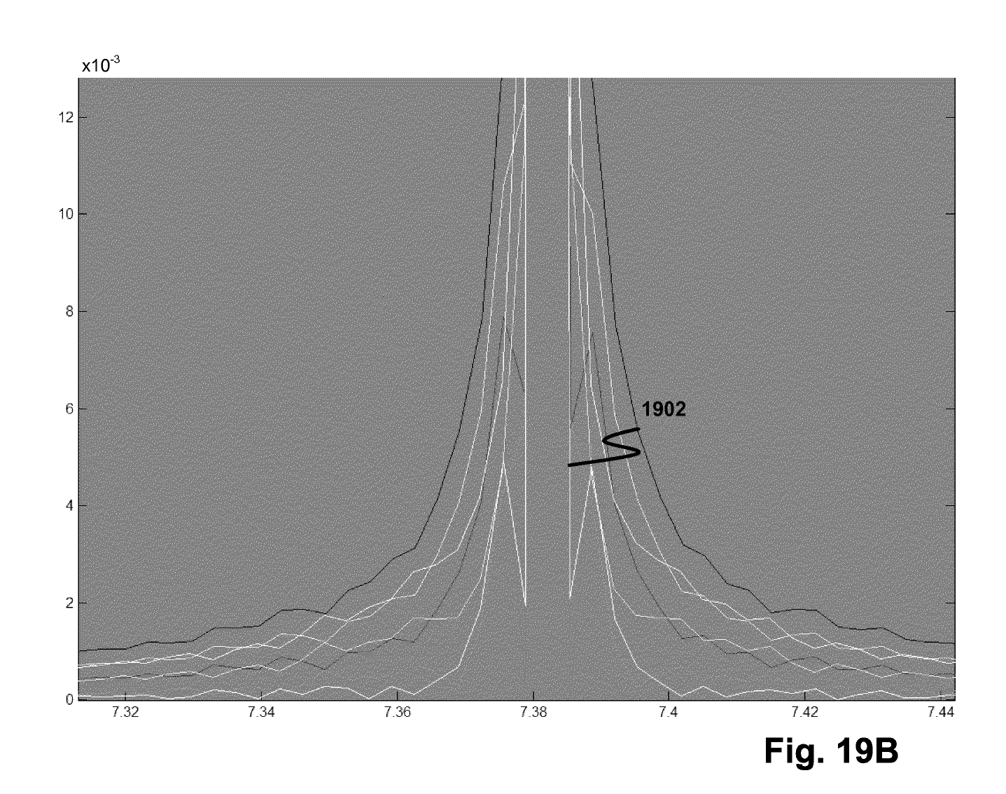

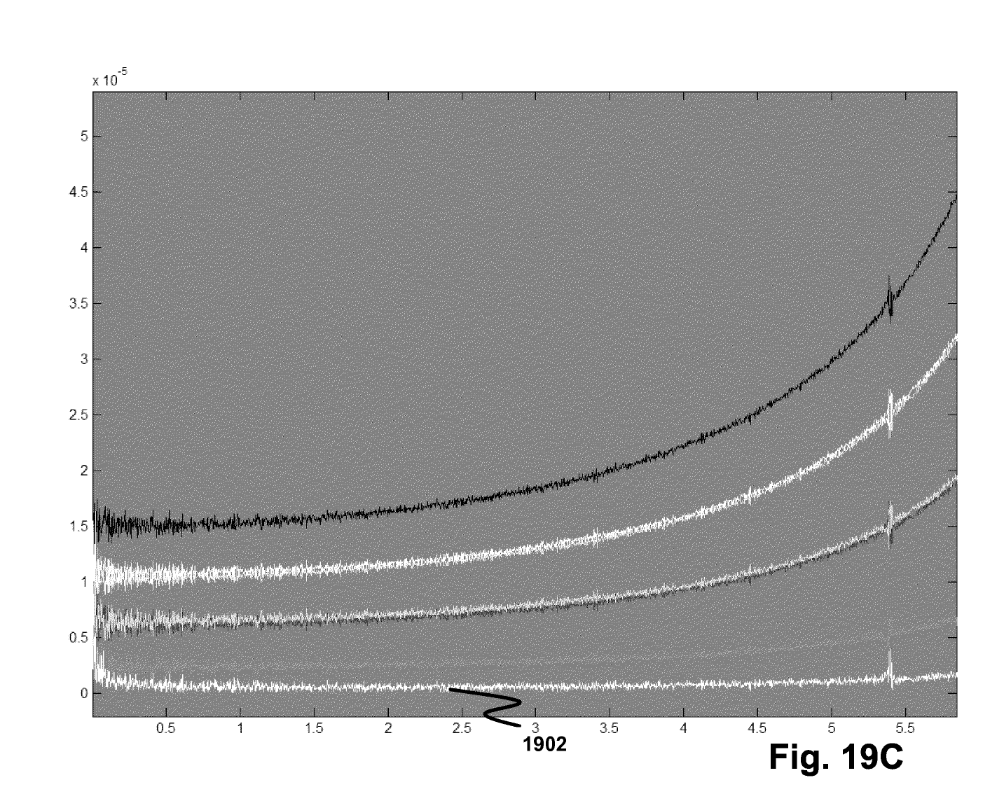

When analyzing evoked activity, spontaneous activity can be considered noise. Recording of the stimulus in evoked experiments is used to calculate the precise spectrum of the stimulus. In one implementation, the harmonics of the auditory stimulus can be used to identify frequencies to localize. The tuning of the frequency grid can be done by cutting the time of registration T to build an optimal approximation of the basic frequency and its harmonics. FIGS. 19A-19C illustrate the optimization of a stimulus spectrum in accordance with an illustrative implementation. In these Figures, the optimization of the stimulus spectrum at basic frequency is done by cutting the time T. Notice, that T in FIGS. 19A-19C is measured in number of time steps (each step is equal to 0.002 seconds) and the optimal spectrum 1902 is calculated for the fractional step, based on the precise Fourier transform. FIGS. 19A and 19B illustrate the tops and bottoms of the peaks, respectively, at the basic frequency 7.3821 Hz. FIG. 19C illustrates the plateaus that can occur at low frequencies. The optimal T, found in FIG. 19C, can be used to calculate the multichannel spectra of MEG. Using the optimal T ensures that the energy of the response is perfectly concentrated in stimulus harmonics, giving the best possible coherence. The parameter T can be optimized for any selected frequency, e.g., for frequency of some spontaneous activity. In experiments with stimulus parameter T is optimized for stimulus frequency, then the multichannel spectrum is calculated once using the optimized T. This provides the correspondence between the frequency of stimulation and the response frequency. FIG. 18 is a graph of Precise Tuned Fourier spectrum for the stimulus used in the above experiment in accordance with an illustrative implementation. The graph 1800 shows only the odd harmonics but not the even harmonics of the auditory stimulus due to the stimulus profile used in the particular experiment. It also illustrates the high accuracy of the method: results of calculations coincide with theoretical expectations. The response, however, may not be as symmetric in time, so the response can contain all harmonics.

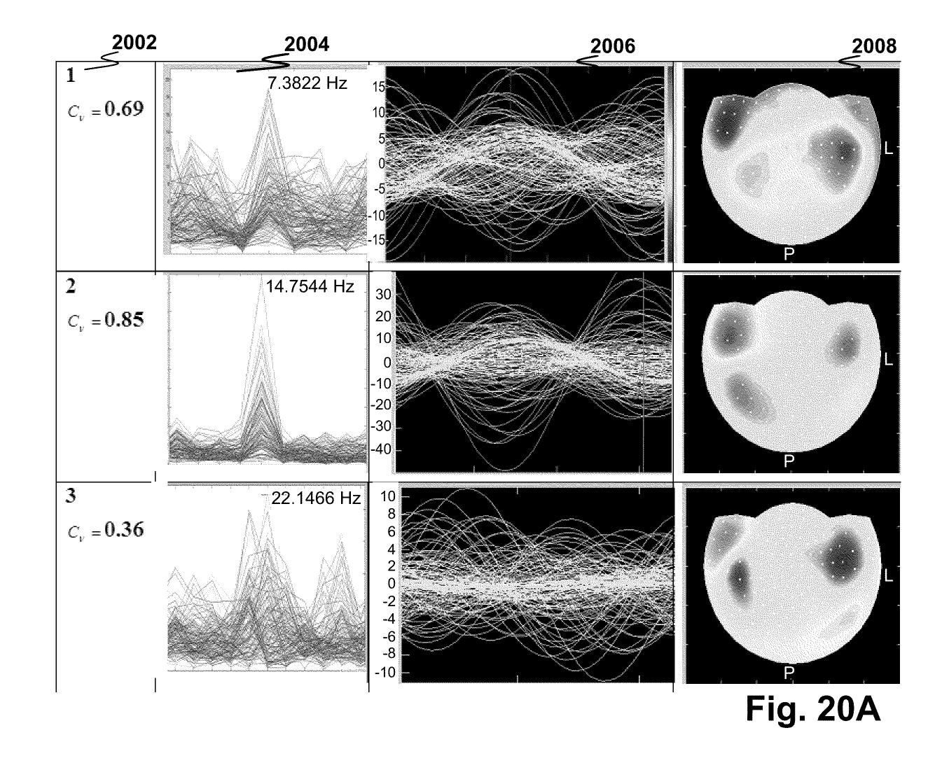

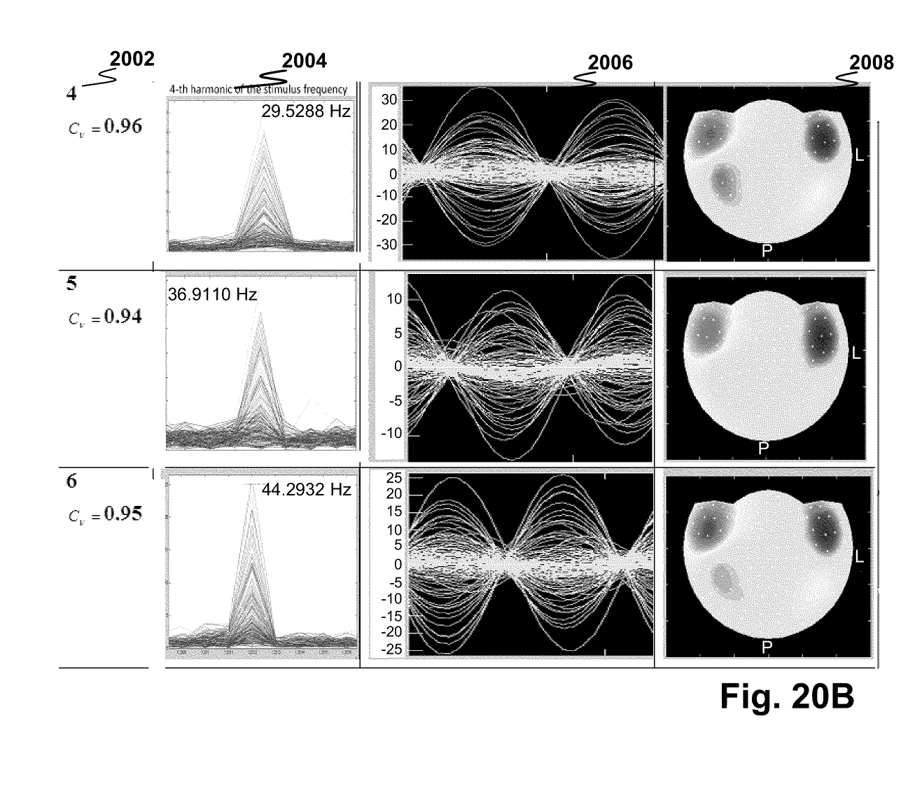

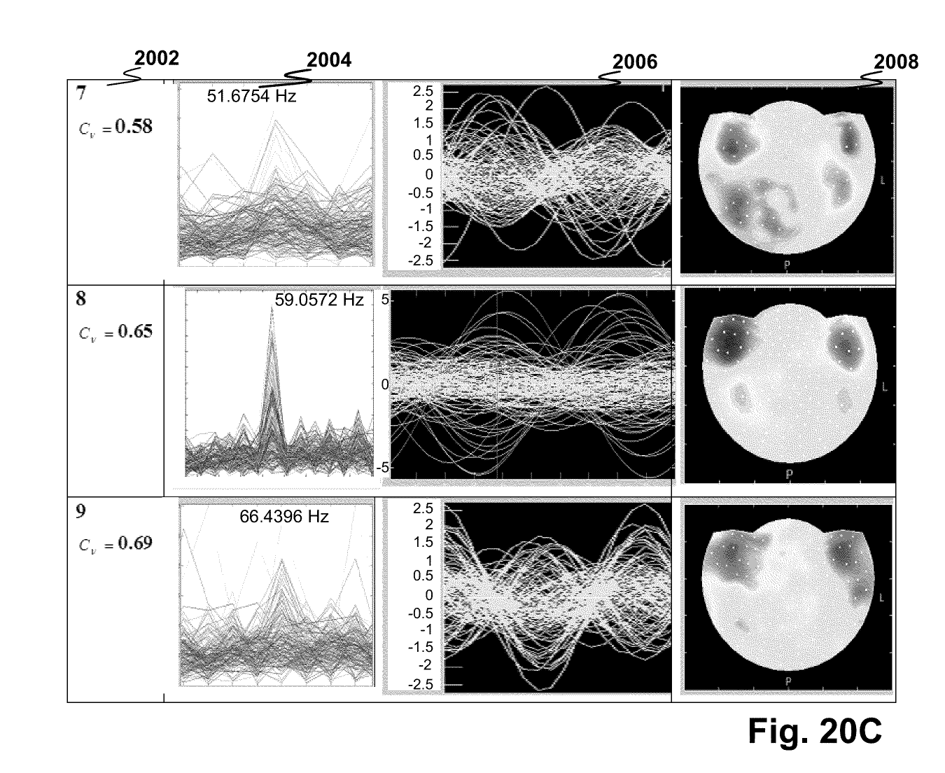

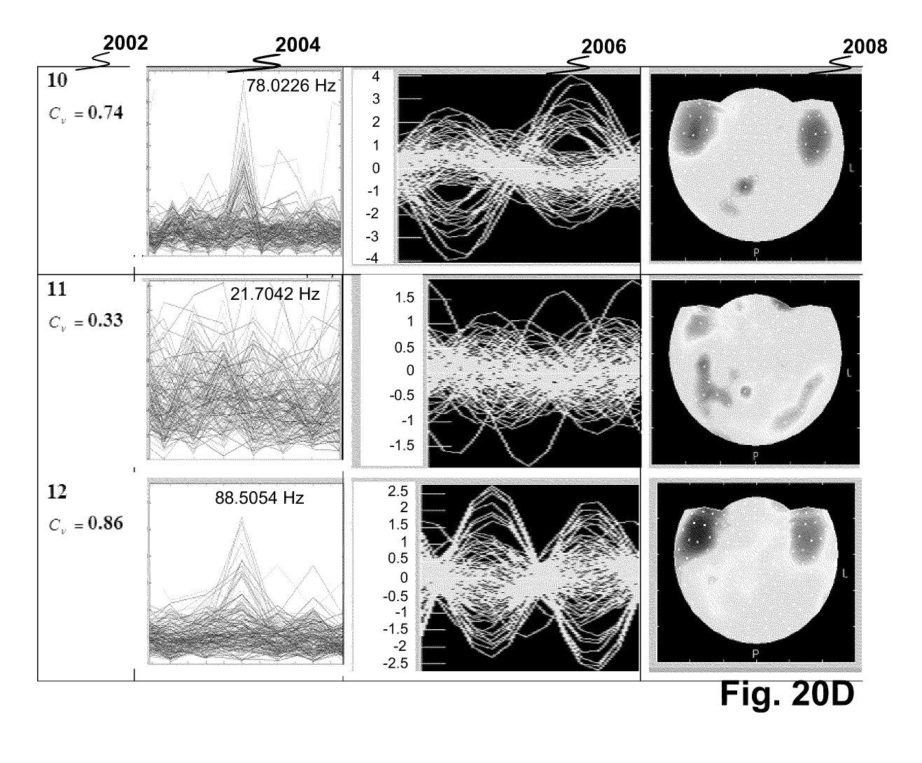

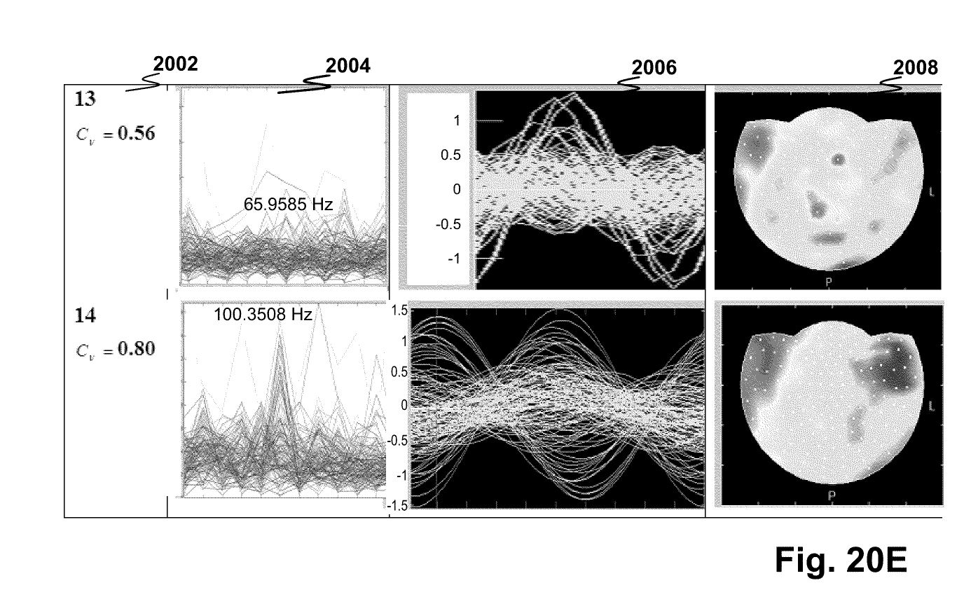

FIGS. 20A-20E illustrate the detailed analysis of the response multichannel spectrum at the stimulus harmonics. Column 2002 includes the number and coherence of the stimulus harmonic. The precise multichannel spectrum for the frequency band near the harmonic is shown in column 2004. The difference in amplitude between the harmonic and neighboring frequencies illustrates noise reduction. The time courses of the reconstructed signals in all channels for the period of the harmonic are shown in column 2006. The magnetic field pattern at the moment of maximal instantaneous power is shown in column 2008. Intensity of the shading corresponds to the amplitude of the induction, with darker regions corresponding to larger amplitudes. The sensors, shown as white squares, are also shown in the magnetic field patterns. Using the data in FIGS. 20A-20E, the distribution of the response energy between harmonics can be found. In addition, the precise frequency-pattern analysis allows the response to be detected even at very high frequencies. Some harmonics, e.g., 4, 5, and 6, produce similar patterns. The time course for the structure that produces the patterns can therefore be found. This is true, even though not all patterns are similar. Differing patterns can mean that the auditory response is produced by different structures. Finally, and as described in greater detail below, the analysis of the 8^{th} harmonic was used to reveal the moving sources corresponding to possible pathways of the auditory nervous signal.

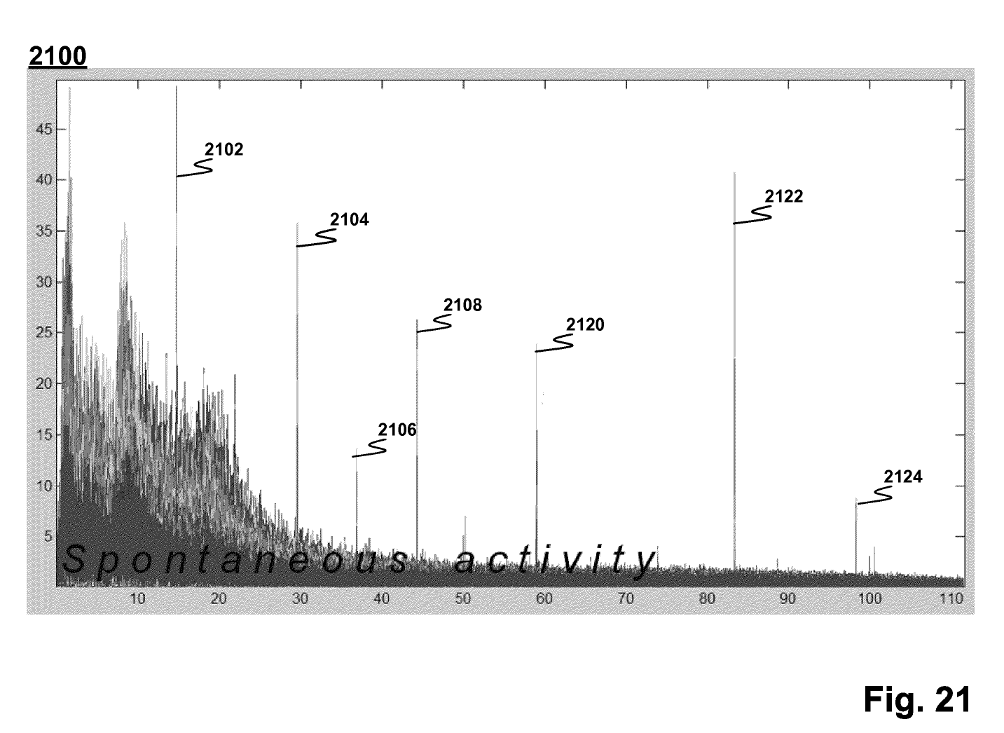

As noted above, the response is not as symmetric in time compared to the auditory stimulus. Accordingly, all harmonics of the auditory stimulus can be analyzed. FIG. 21 is a graph of precisely tuned multichannel spectrum of measured magnetic fields in accordance with an illustrative implementation. Various spikes in the graph 2100 can be identified as potentially useful frequencies that correspond with a harmonic of the auditory input or as noise. For example, in one implementation, signals 2122 and 2124 can be ignored as noise since they do not correspond to a harmonic of the auditory input. Spikes 2102, 2104, 2106, 2108, and 2120, may correspond with the 2^{nd}, 4^{th}, 5^{th}, 6^{th}, or 8^{th} harmonic, respectively, and therefore, may be interesting. Noise signals, however, may also be near one or more of the spikes. A more detailed view of the frequency grid can be used to isolate useful frequencies from noise.

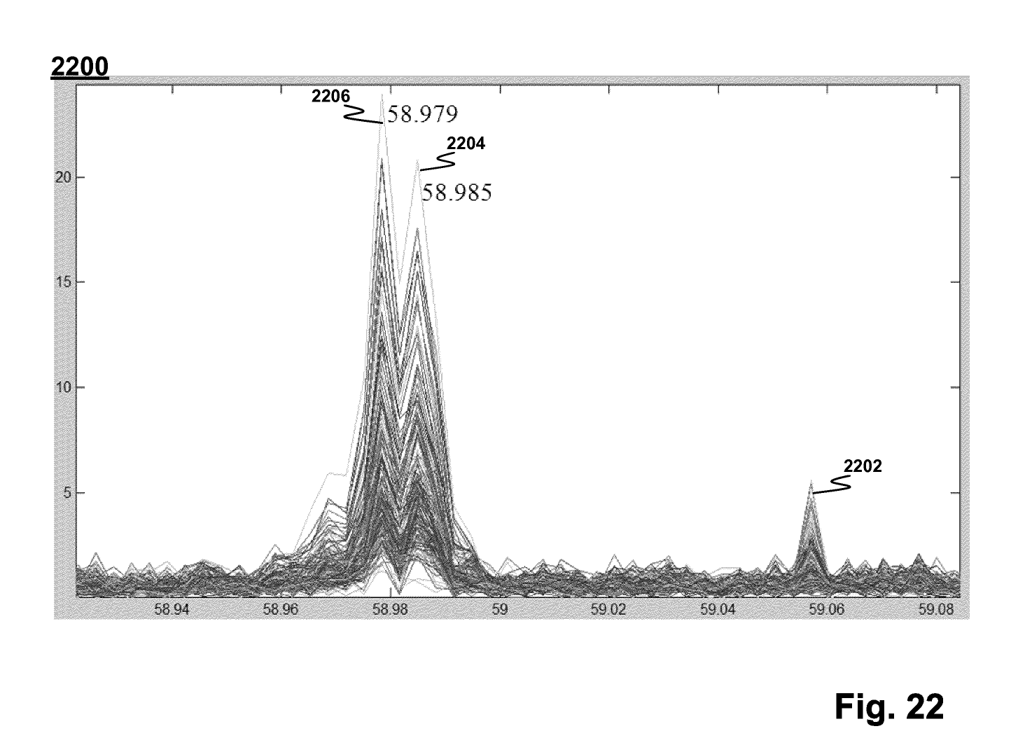

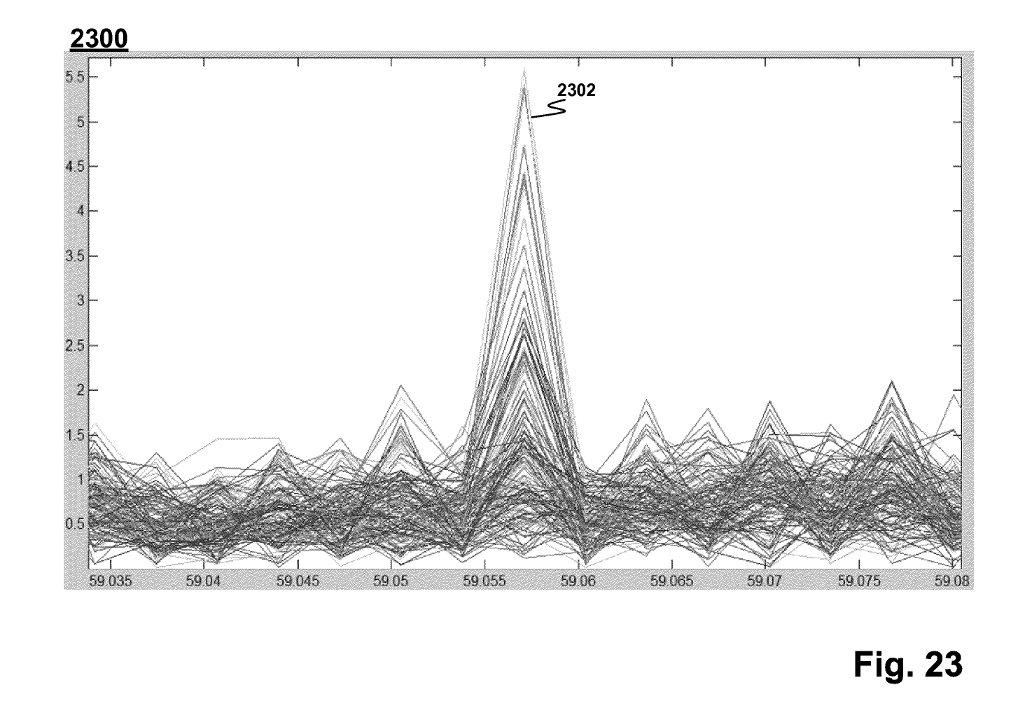

As an example, spike 2120 is near 59 Hz which corresponds to roughly the 8^{th} harmonic of the auditory input, as well as the frequency of alternating current in the United States. Accordingly, noise from a power source may be contributing to the spike 2120. A more detailed view of the frequency grid can be used to separate a useful frequency from noise from a power source. FIG. 22 is a graph of measured magnetic fields spectra in a narrow frequency range that includes the 8^{th} harmonic of the stimulus in accordance with an illustrative implementation. Graph 2200, which is based upon the analysis of the experimental data using the mathematical formulas as described above, shows an interesting spike 2202 that directly corresponds to the 8^{th} harmonic of the auditory input, i.e., ˜59.06 Hz. Graph 2200 also allows the identification of spikes 2204 and 2206, which correspond to unwanted noise from power grid. FIG. 23 is a graph of measured magnetic fields spectra in a narrow frequency range that includes the 8^{th} harmonic of the stimulus in accordance with an illustrative implementation. Graph 2300 is an even more detailed view of frequency grid that shows spike 2302 that corresponds to the 8^{th} harmonic of the auditory input, e.g., 59.0568 Hz. Looking at the frequency grid in different scales allows to distinguish between noise and useful signals in order to localize the latter.

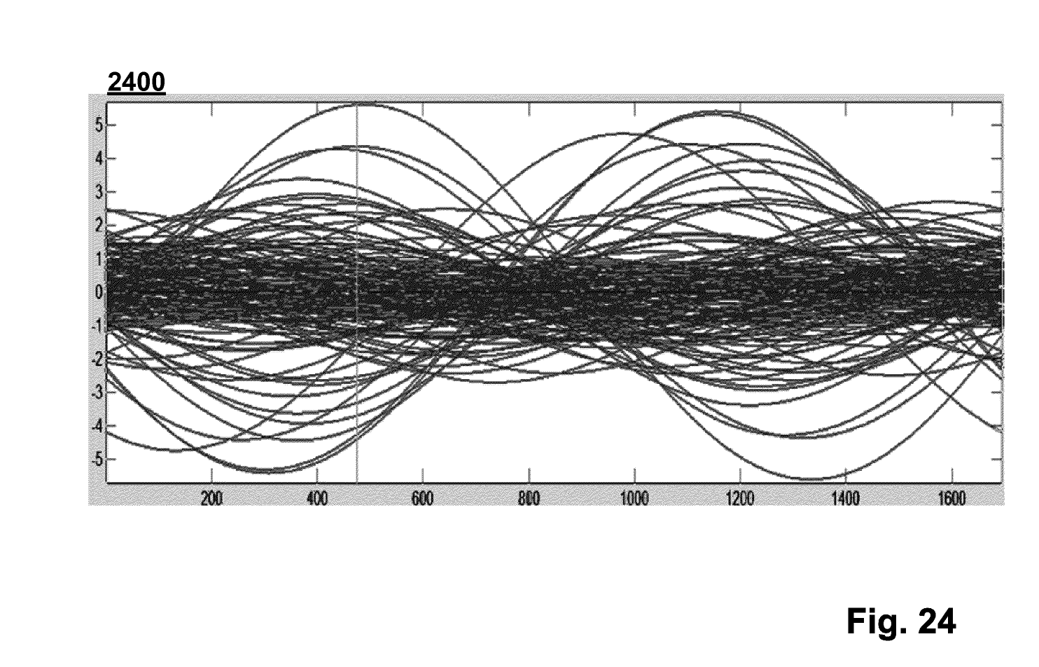

The inverse Fourier transform for the selected harmonics can then be calculated. FIG. 24 is a graph that illustrates phase shifts between channels at the frequency of the 8^{th} harmonic in accordance with an illustrative implementation. The phase shifts between channels lead to the movement of sources of magnetic fields. This estimation of the resulting multichannel time series of MEG data allows for the tracking of movement of the source of the magnetic fields over time.

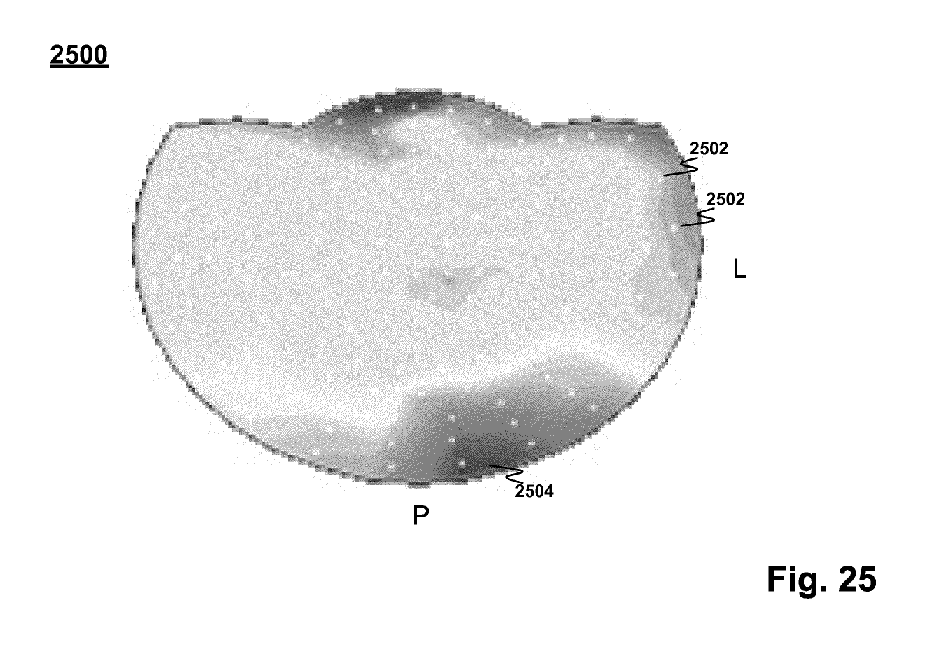

As described above, particular frequencies corresponding to spikes in a frequency grid can be selected for use in localizing the source of magnetic field, restored at the spike frequency. Accordingly, the ability to tell noise from a non-noise signal is beneficial. A noise signal is simply a signal that is not to be used in localizing magnetic fields. Depending on the type of analysis being done, a signal may or may not be noise. For example, during spontaneous activity analysis, magnetic fields external to the brain can be considered noise. A spontaneous magnetic field, however, may be considered noise during evoked activity analysis if the spontaneous field does not correspond with a harmonic of the auditory input. Magnetic fields can be identified as non-characteristic for human magnetic fields, if classified as a magnetic field that is external to the brain. As an example, an inverse Fourier transform can be done across all channels at a frequency of 58.979 Hz. As shown in FIG. 22, spike 2206 corresponds with a noise signal—the supposed power grid frequency. The inverse Fourier transform produces data that allows the estimation and localization of the magnetic field to be determined. FIG. 25 illustrates a noise magnetic field in accordance with an illustrative implementation. The noise magnetic field 2500 shows the spatial distribution of the magnetic field across a helmet used to collect the data from sensors 2502. The field maxima 2504 are located near the helmet border, which indicates that the magnetic field is external to the head. Accordingly, this magnetic field can be ignored in the analysis of the data.

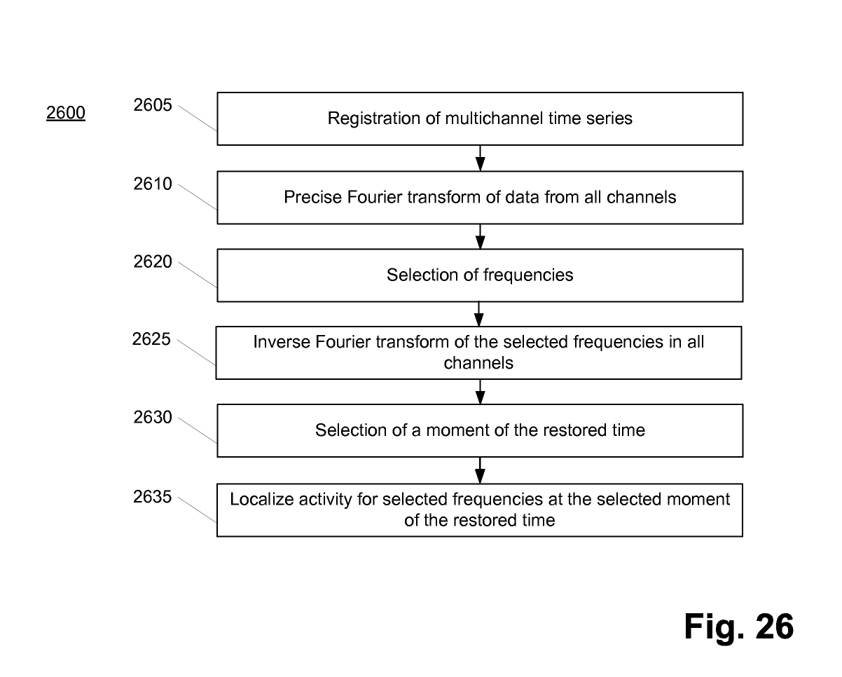

FIG. 26 is a flow diagram for spontaneous field analysis in accordance with an illustrative implementation. The process 2600 can be implemented on a computing device. In one implementation, the process 2600 is encoded on a computer-readable medium that contains instructions that, when executed by a computing device, cause the computing device to perform operations of process 2600.

Data is collected for a period of time across multiple channels, each of which corresponds to a magnetic field recording device. The data is used for the registration of a multichannel time series (2605). A precise Fourier transform of the data from all channels is done (2610). The transform translates the data into the frequency domain. In one implementation, the multichannel frequency spectrum can be displayed. Based upon the multichannel frequency spectrum, one or more frequencies of interest can be selected (2620). For example, any frequency corresponding to a spike in the multichannel frequency spectrum can be selected. An inverse Fourier transform can be done for each of the selected frequencies (2625). A moment of restored time is selected, giving a field pattern (2630). An inverse problem solution can be used to localize brain activity or external noise for the selected frequency at the selected moment of the restored time (2635).

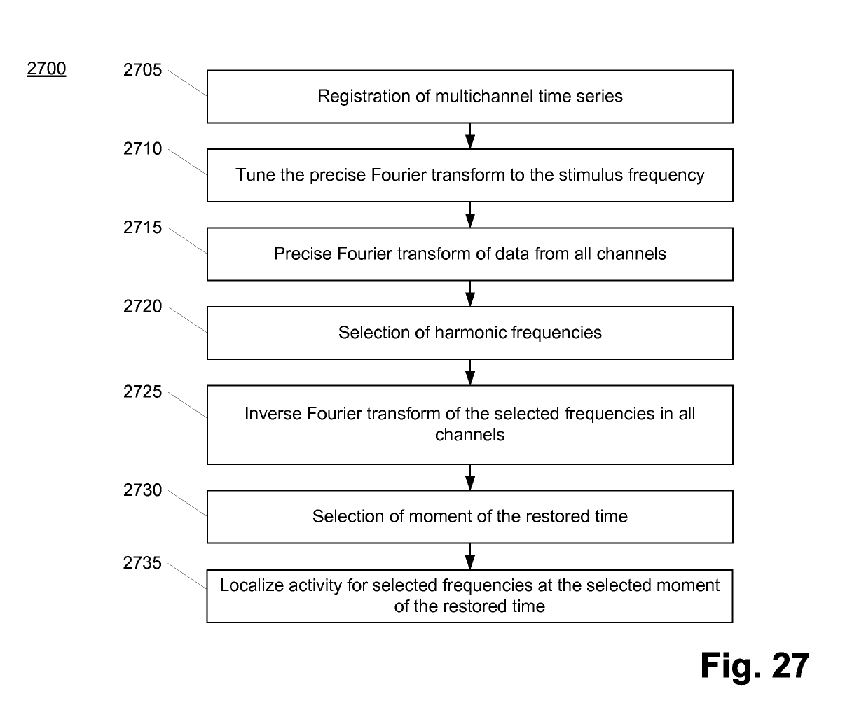

FIG. 27 is a flow diagram for evoked field analysis in accordance with an illustrative implementation. The process 2700 can be implemented on a computing device. In one implementation, the process 2700 is encoded on a computer-readable medium that contains instructions that, when executed by a computing device, cause the computing device to perform operations of process 2700.

Data is collected for a period of time across multiple channels that each correspond to a magnetic field recording device. During the period of time, a stimulus of a known frequency is provided to a subject. The brain activity is used for the registration of a multichannel time series (2705). A precise Fourier transform is tuned to the stimulus frequency (2710). The precise tuned Fourier transform of the data from all channels is done (2715). The transform translates the data into the frequency domain. In one implementation, one or more harmonic frequencies of the auditory stimulus can be selected (2720). An inverse Fourier transform can be done for each of the selected frequencies (2725). A moment of restored time is selected, giving a field pattern (2730). An inverse problem solution can be used to localize evoked brain activity for the selected frequency at the selected moment of the restored time (2735).

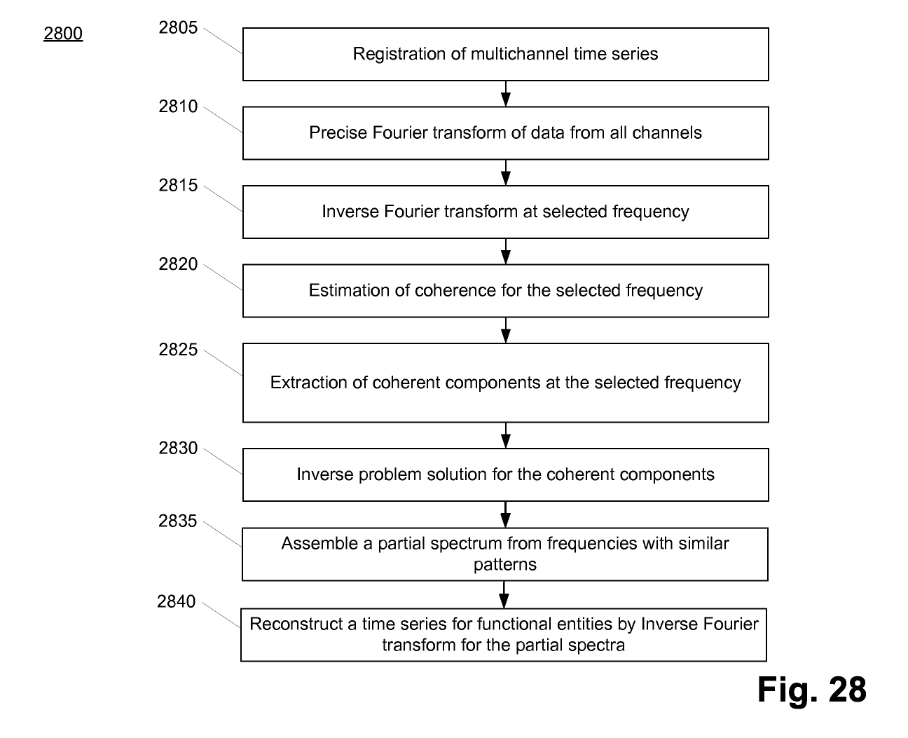

FIG. 28 is a flow diagram for spontaneous field analysis in accordance with an illustrative implementation. The process 2800 can be implemented on a computing device. In one implementation, the process 2800 is encoded on a computer-readable medium that contains instructions that, when executed by a computing device, cause the computing device to perform operations of process 2800.

Data is collected for a period of time across multiple channels, each of which corresponds to a magnetic field recording device. The data is used for the registration of a multichannel time series (2805). A precise Fourier transform of the data from all channels is done (2810). The transform translates the data into the frequency domain. A selected frequency is received, for example, through a user interface. An Inverse Fourier transform is done at the selected frequency (2815). An estimation of the coherence for the selected frequency can then be determined (2820). Coherent components at the selected frequency are extracted (2825). An inverse problem solution from the coherent components can then be calculated (2830). A partial spectra from frequencies with similar patterns can be assembled (2835). A time series for functional entities can be reconstructed by an Inverse Fourier transform of the partial spectra (2840).

### Analysis of the MEG Partial Spectra for Thalamic Brain Activity

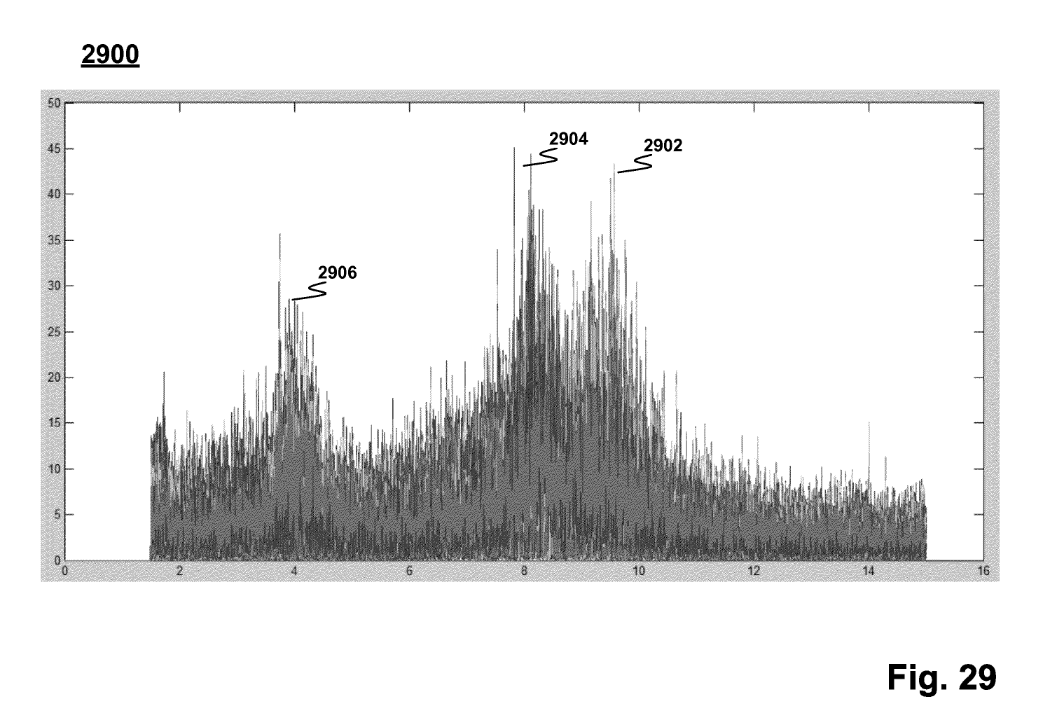

Data can be collected by monitoring a number of multiple sensors place over a body or space. The sensors can record signals generated by components within the body or space. As described above, the recorded signals can be used to generate a multichannel signal that is represented as a sum of elementary coherent oscillations. A partial spectra analysis can be done on the elementary coherent oscillations. A step of the partial spectra analysis is to define the set of frequencies to be studied. In this example, a partial spectra was extracted from the frequency band 1.5-15 Hz of the experimental spectrum of a subject with thalamo-cortical dysrhythmia. FIG. 29 shows a general view of the multichannel spectrum 2900 in this band, giving the total decomposition of the MEG to functionally invariant entities.

This spectrum 2900 contains the peak of alpha activity (9-10 Hz) 2902 and several peaks 2904 and 2906 of pathological theta activity (4-8 Hz). After the precise frequency-pattern analysis is done as described above, this multichannel spectrum can be represented as a sum of 8000 elementary coherent oscillations. In other implementations, the multichannel spectrum can be represented by greater or fewer elementary coherent oscillations. Each elementary oscillation has distinct frequency, constant pattern and is produced by the functional entity having a constant spatial structure. Determining which oscillations to analyze is not trivial.

The magnetic induction from a dipole Q can calculated as described above. Specifically, the following formulas can be used:

$$B ( r ) = - {μ}_{0} ∇ U ( r ) , U ( r ) = - \frac{1}{4 π} \frac{( Q \times {r}_{0} , r )}{F}$$

$$F = a ( ar + {r}^{2} - ( {r}_{0} , r ) ) , a = r - {r}_{0} , {μ}_{0} = 4 π \cdot {10}^{- 7} .$$

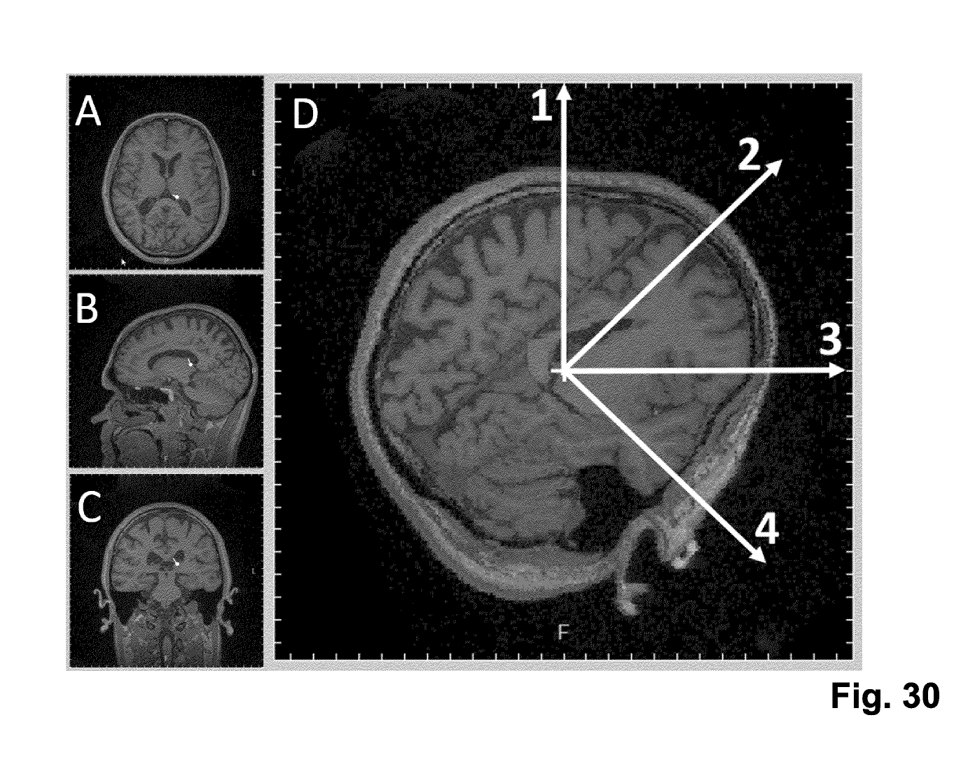

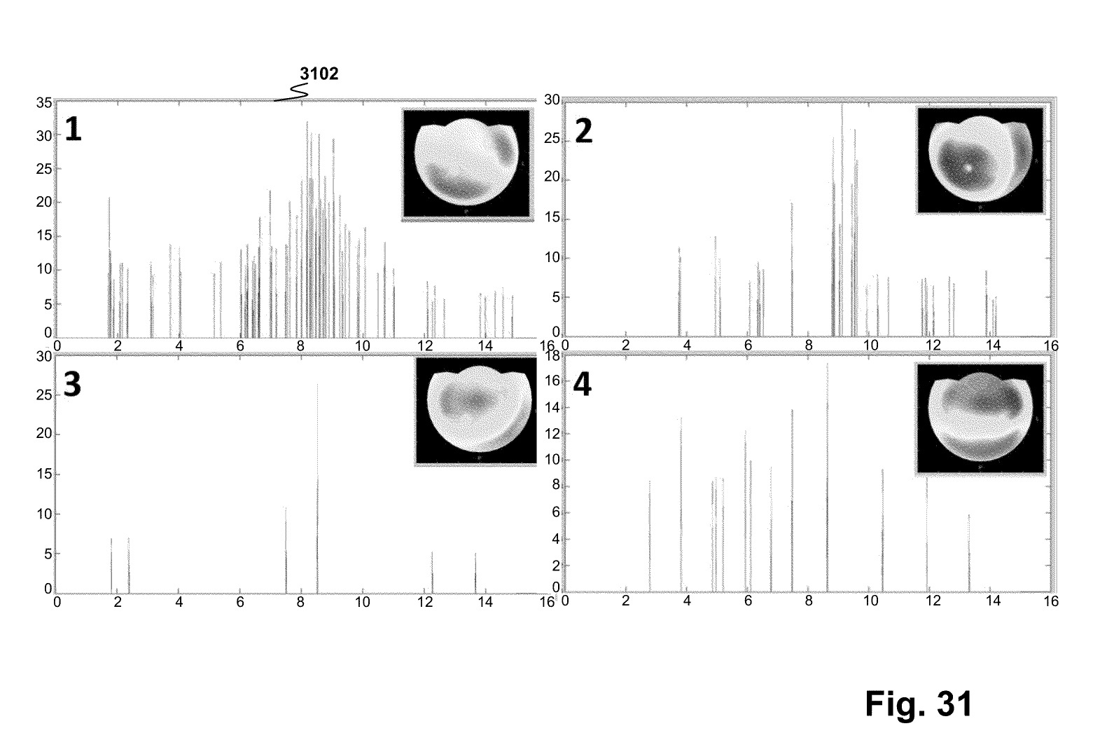

In one implementation, all trial dipoles lay in the same plane, orthogonal to r₀, because the vector product Q×r₀ is nonzero only for those dipoles. FIG. 30 illustrates the location and directions of a trial dipole. Panels A-C of FIG. 30 show the common position of the trial dipole. Direction of a signal from this dipole can be calculated. In one implementation, each trial dipoles cover 360 degrees by eight directions with 45 degree steps. Panel D of FIG. 30 shows the directions of possible trial dipoles. Note that only four directions are shown in Panel D of FIG. 30, because the other four correspond to reflection of first four and produce field patterns, differing only in sign, not form. In other implementations, finer granular steps can be used. For example, 16 directions with 22.5 degree steps, 36 directions with 10 degree steps, etc. can be used.

FIG. 31 shows four normalized patterns, produced by the trial dipole. Also the corresponding partial spectra are shown, assembled by the algorithm described above. Note that partial spectra differ for different dipole directions, even if the dipole have common location.

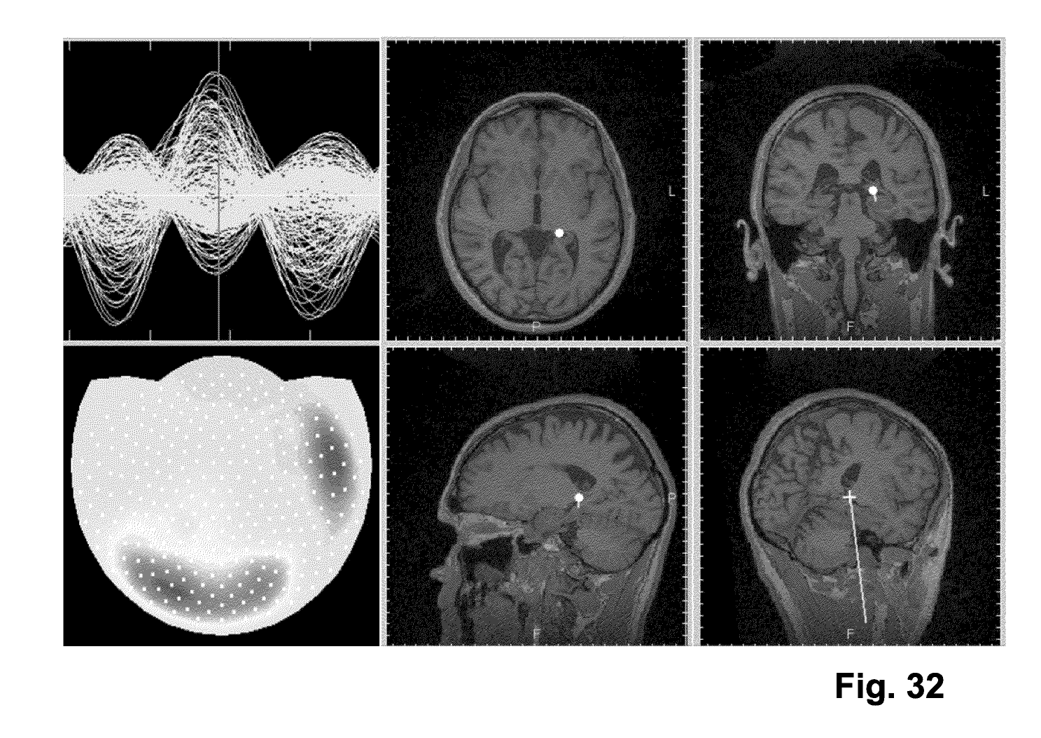

FIG. 32 illustrates the reconstruction of the time dependence of magnetic fields from the partial spectrum 3102 and the inverse problem solution to estimate spatial structure. These fields are generated by the functional entity, oscillating as a whole and having the spatial structure, estimated from the shown field pattern.

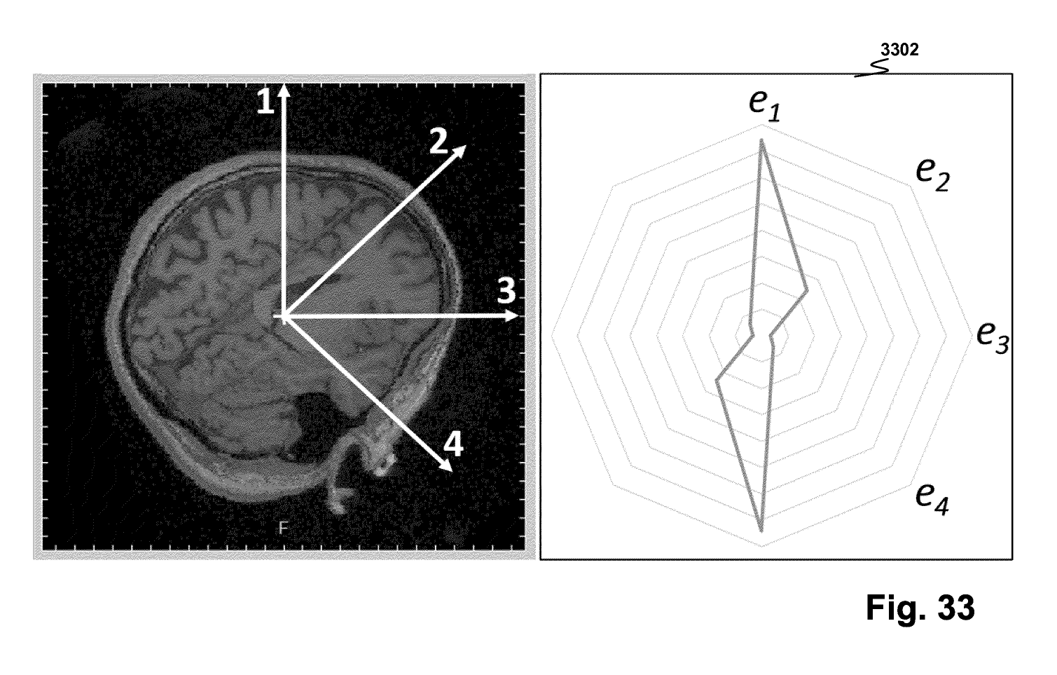

FIG. 33 shows the distribution of the total energy between the different directions of a trial dipole. Energy, generated in each direction, was obtained by the summation of the partial power spectrum:

$${E}^{tr} = {f}^{2} ( {E}_{10} ) {D}_{10}^{2} + {\sum }_{n = 1}^{N} {\sum }_{m = 1}^{M} {f}^{2} ( {E}_{mn} ) {D}_{mn}^{2} .$$

Relative energy for each direction was calculated by eᵢ=E^{tri}/(E^{tr1}+E^{tr2}+E^{tr3}+E^{tr4}) and plotted as the angular intensity curve 3302.

It can be concluded, that oscillation of the dipole in direction 1 is dominant for this trial location. Calculating these dominant directions on the grid of trial dipoles positions, it is possible to build the vector field of current directions. From this field, the path of a signal through a body can be estimated. For example, this approach can be used to define the possible directions of neuronal circuits or the functional configurations of brain structures.

### Functional Tomography of the Human Body Based on Multichannel Magnetic Measurements

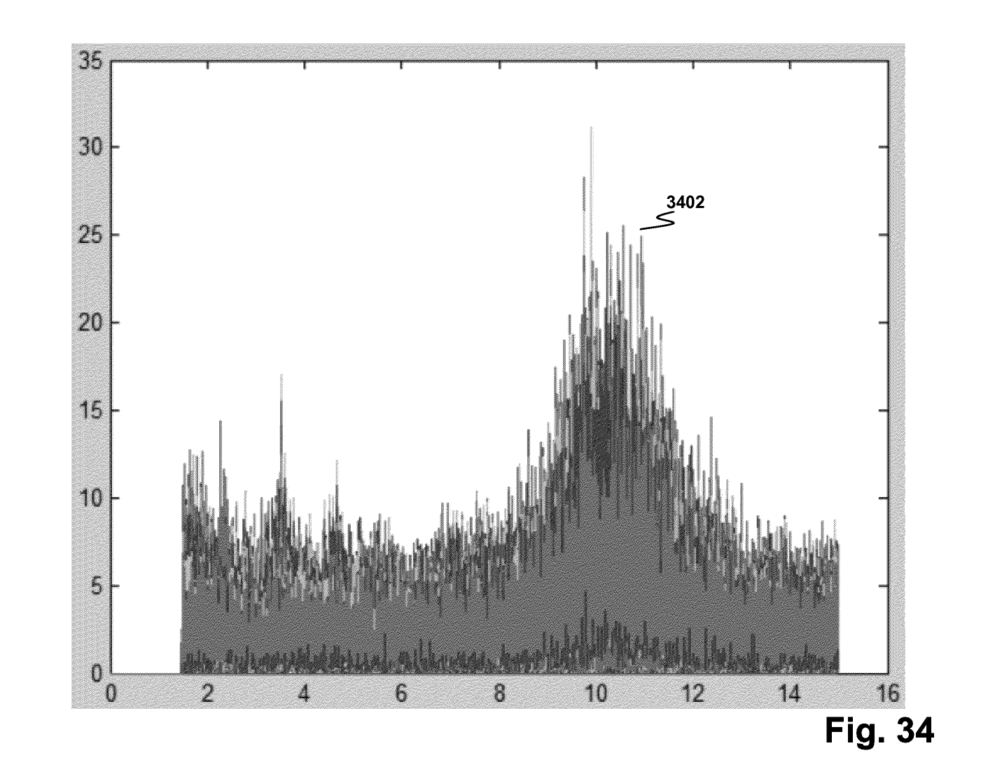

Data can be collected by monitoring a number of multiple sensors place over a body or space, such as a subject's brain, heart, hand and other parts. Also the whole body can be measured with appropriate set of sensors. The sensors can record signals generated by components within the body or space. As described above, the recorded signals can be used to generate a multichannel signal that is represented as a sum of elementary coherent oscillations. A partial spectra analysis can be done on the elementary coherent oscillations. A step of the partial spectra analysis is to define the set of frequencies to be studied. In this example, the subject's head was measured by the set of 275 sensors of the magnetic field. A partial spectrum was extracted from the frequency band 1.5-15 Hz of an experimental spectrum of a control subject. FIG. 34 shows the general view of the multichannel spectrum in this band, giving the total decomposition of the MEG to functionally invariant entities. This spectrum contains the peak of alpha activity (9-11 Hz) 3402. After the fourth step of the precise frequency pattern analysis described above, this multichannel spectrum is represented as a sum of 8000 elementary coherent oscillations. In other implementations, the multichannel spectrum can be represented by greater or fewer elementary coherent oscillations. Each elementary oscillation has distinct frequency, constant pattern and is produced by the functional entity having a constant spatial structure. Determining which oscillations to analyze is not trivial.

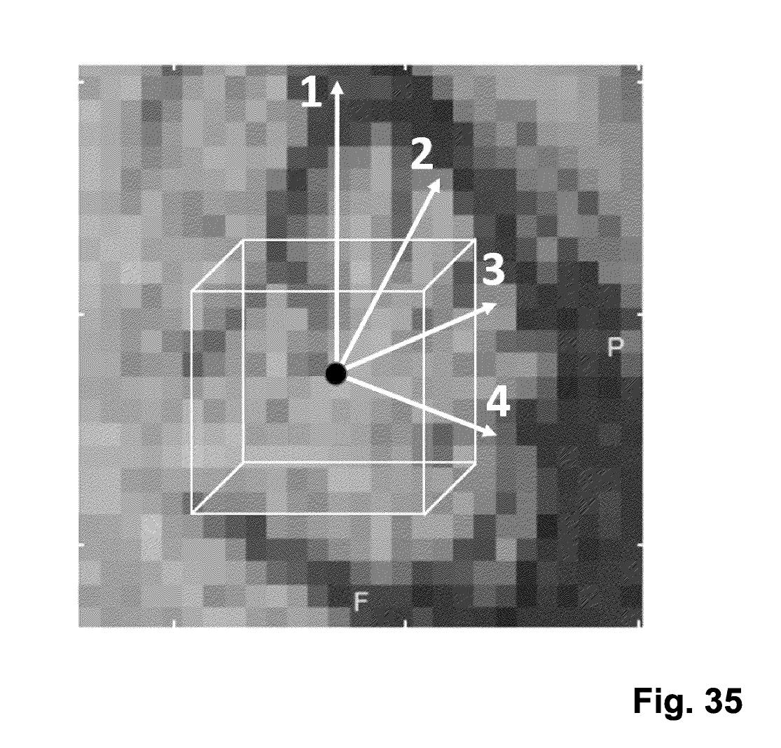

A functional tomogram displays a 3-dimensional map of the energy, produced by all sources, located at a given point. In order to build a functional tomogram, in the current example a space is determined as a cube 256 mm×256 mm×256 mm, including the subject's head. This cube is divided into Nₓ×N_{y}×N_{z} elementary blocks. The edge of the blocks is selected in accordance with desirable spatial resolution, in this illustrative embodiment it is equal to 1 cm. To calculate the energy, produced by all sources, located in the center of the block, a set of trial dipoles can be built. FIG. 35 shows the elementary block of the functional tomogram and the directions of the trial dipoles. The number of trial dipoles can vary and depends on the desirable precision. In this example, the number of trial dipoles is equal to 4. In other examples, the number of trial dipoles could be more or less.

Trial field normalized patterns can be generated and partial spectra can be assembled as described above. A functional tomogram of the space monitored by the sensors can be generated based on the partial spectra. The space can be divided into a number of blocks, such as cubes, voxels, etc. The total energy for a block ijk can be calculated as:

E^{ijk}=E^{tr1}+E^{tr2}+E^{tr3}+E^{tr4}.

Each energy E^{tr} is calculated as summation of energy produced by the functional entity, corresponding to one of the trial patterns:

$${E}^{tr} = {f}^{2} ( {E}_{10} ) {D}_{10}^{2} + {\sum }_{n = 1}^{N} {\sum }_{m = 1}^{M} {f}^{2} ( {E}_{mn} ) {D}_{mn}^{2} .$$

The energy for each block can be calculated as:

E^{ijk} for i=1, . . . ,Nₓ;j=1, . . . ,N_{y};k=1, . . . ,N_{z}.

Based upon the calculated energy for the blocks, the functional tomogram can be obtained. A functional tomogram is the 3-dimensional function, giving the spatial distribution of the general MEG recorded energy of the frequency band. Several methods to utilize the functional tomogram are described below.

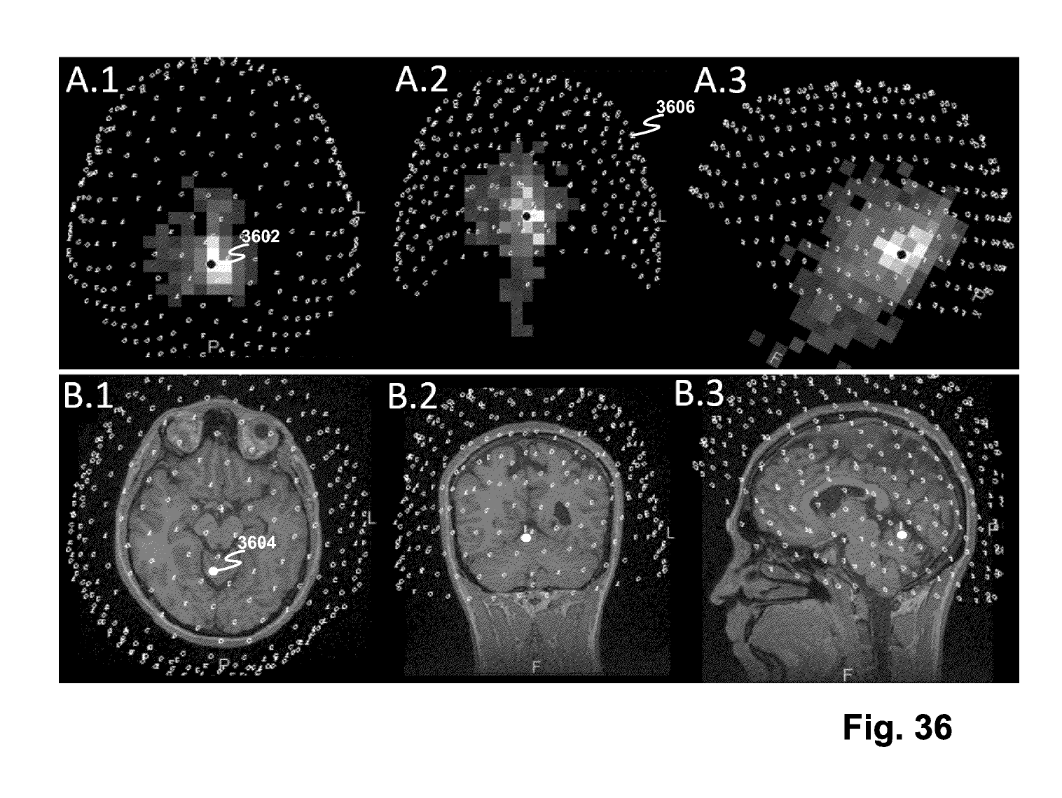

FIG. 36 shows a functional tomogram and an MRI of a subject's head in the same experiment. Corresponding slices of the both tomograms are shown, going through the same point in space. This point is depicted by the black circle 3602 in the functional tomogram, and by the white circle 3604 in the MRI. Sensor positions are shown by small white circles, for example, white circle 3606. The functional tomogram along with the MRI can be utilized in the following ways:

1. The position in space of the MRI (B.1-3) can be selected, corresponding to some anatomical detail of the brain. The same position in space of the functional tomogram (A.1-3) will show relative activity of this detail in the given frequency band.

2. Selection of a detail in space of the functional tomogram (A.1-3) can be used to determine the anatomical meaning of this phenomenon by finding the corresponding position in space of the MRI.

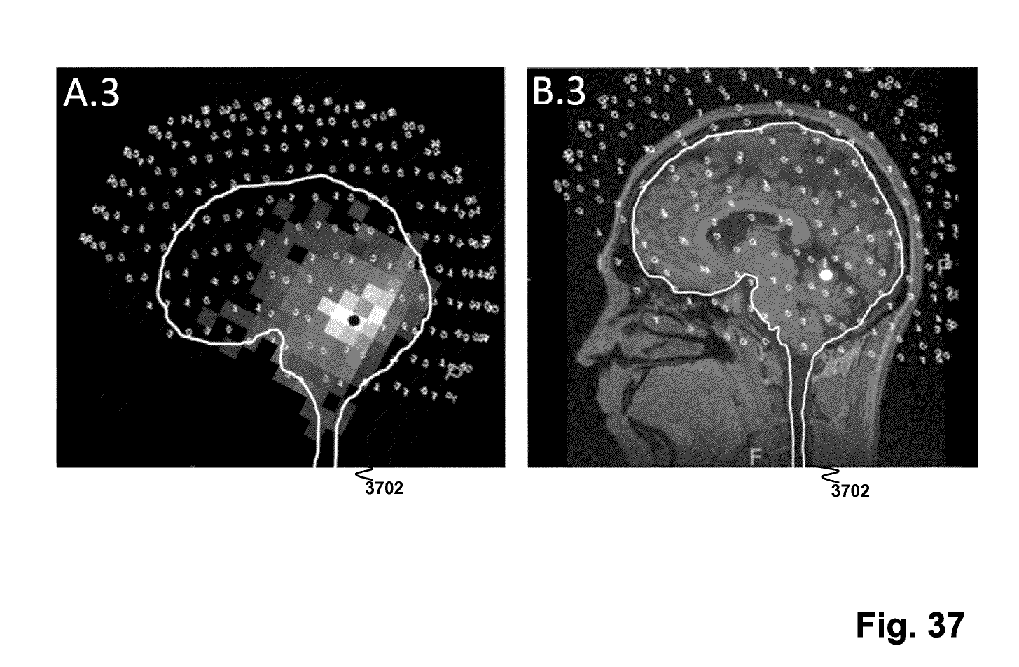

3. Many elements of the functional tomogram, corresponding to the anatomical structure, can be selected using different kinds of pointing devices, such as mouse, trackball, stylus, touchscreen etc. For the sources, composing this anatomical structure, the sum of the partial spectra can be calculated. The Inverse Fourier Transform of this sum of spectra makes it possible to study the signal, produced by the structure, or to subtract it from the general signal. FIG. 37 illustrates the selection of the brain (white line), using the B.3 panel from FIG. 36, while the panel A.3 shows the set of brain activity sources. As explained above, the summation of partial spectra from this set provides the spectrum of the brain magnetic encephalogram, cleared from the interference of other sources.

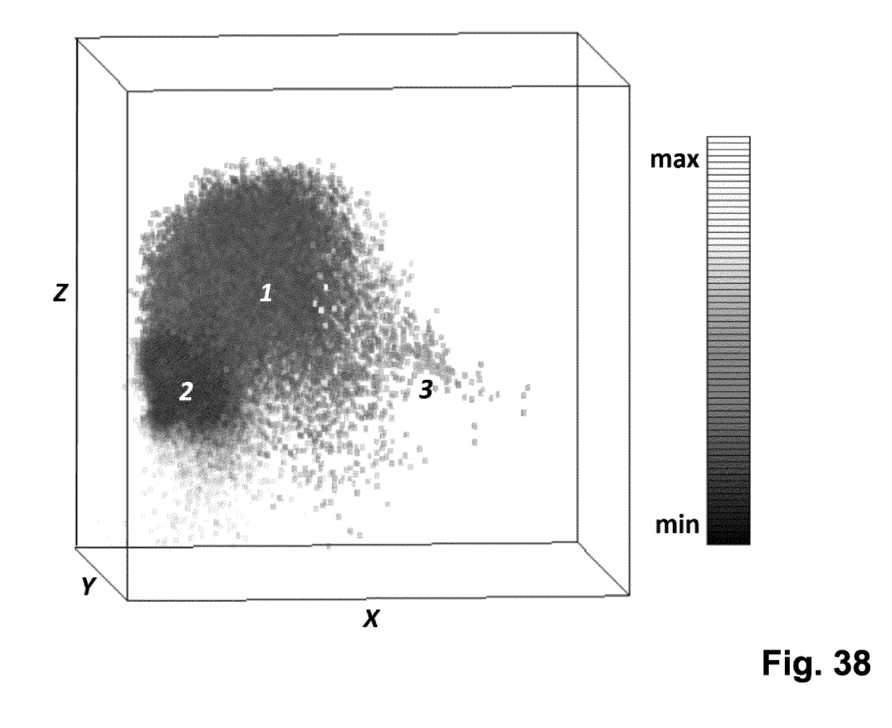

FIG. 38 illustrates a functional tomogram of a brain, neck muscles, and olfactory system in the frequency band 0.003-100 Hz with spatial resolution 3 mm in accordance with an illustrative implementation. Note, that the functional tomogram for the magnetic encephalography data was calculated without any knowledge about the head structure. As can be seen from the FIG. 38, many anatomical details can be reconstructed from the functional tomogram without MRI. In this example, brain (1), neck muscles (2) and olfactory system (3) can be clearly identified. The method of the functional tomography can be used in other problems to reconstruct the unknown inner structure from the multichannel measurements outside the body.

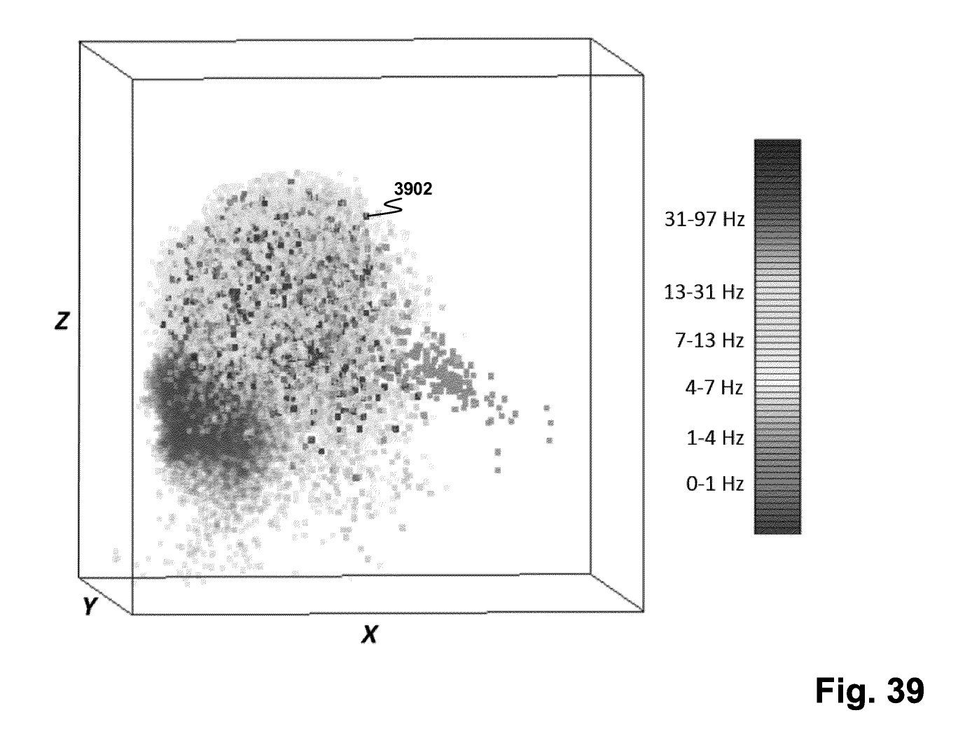

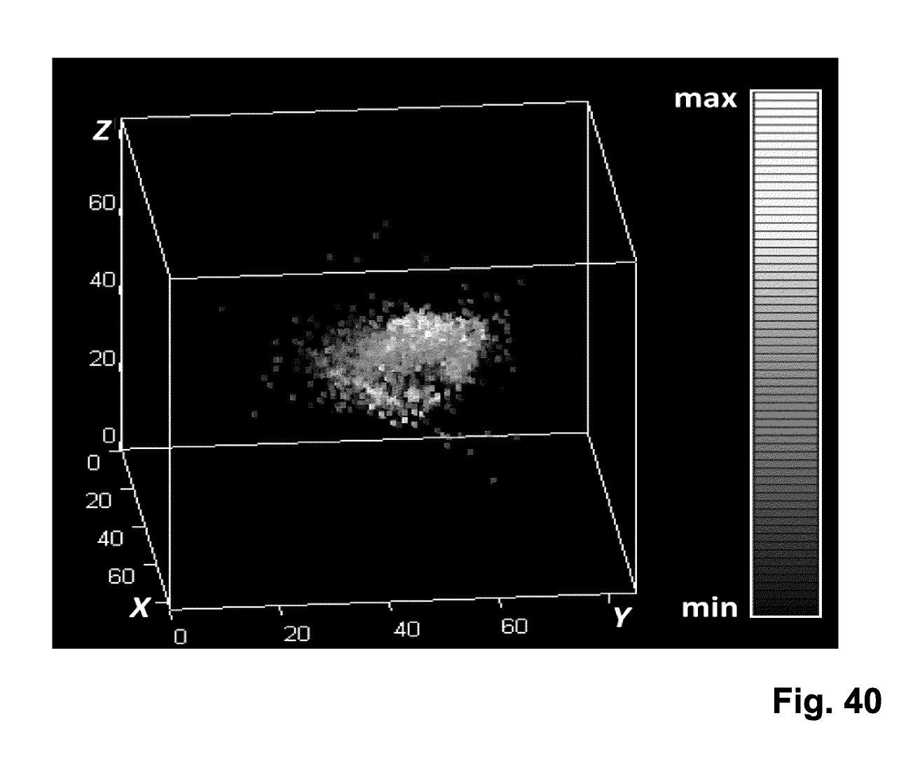

The functional tomography technique can be used to estimate the spatial distribution of the different frequency bands. FIG. 39 shows such distribution for the experiment, shown in FIG. 38, with spatial resolution 3 mm. Spatial structures, corresponding to frequency bands 0-1, 1-4, 4-7, 7-13, 13-31 and 31-97 Hz, are shown. It can be concluded, that different anatomical structures, identified in FIG. 38, produce oscillations in different frequency bands. As shown in FIG. 39, for this particular experiment the neck muscles produce a majority of oscillations in the 31-97 Hz range; the brain produces a majority of oscillations in the 4-31 Hz range; and the olfactory system produces a majority of oscillations in the 0-4 Hz range. Some voxels (shown in black, e.g. 3902) contain sources, oscillating in several bands.

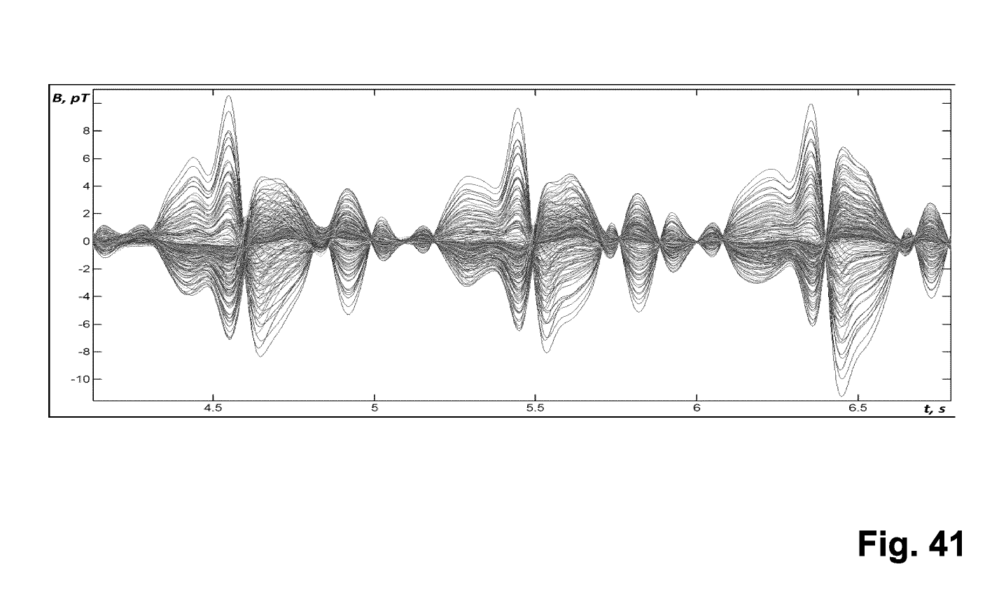

FIG. 40 illustrates a functional tomogram of the human heart in the frequency band 1-12 Hz. Magnetic measurements were performed by means of 275 sensors, located over the chest of the subject. Functional tomogram was calculated with spatial resolution 1 mm in the area of 80×80×80 cubic millimeters. As can be seen from the FIG. 40, the form and size of the reconstructed object correspond to the human heart. FIG. 41 illustrates three cycles of the magnetic cardiogram restored from the partial spectrum of the heart structure, shown in FIG. 40.

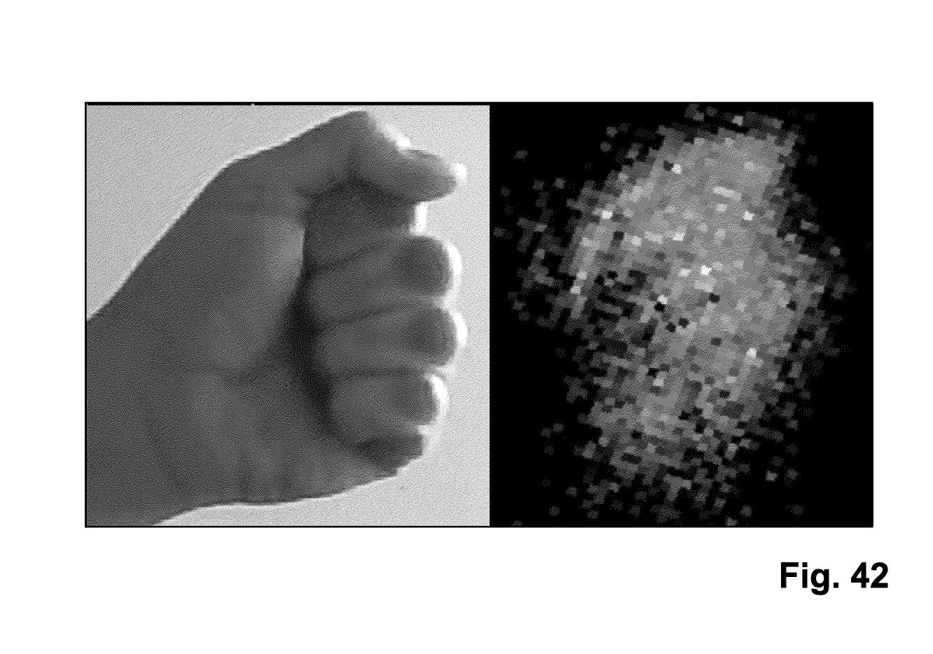

FIG. 42 illustrates a photography of the human hand (clenched fist) and its functional tomogram, calculated from multichannel registration of magnetic field. Functional tomogram was calculated in the frequency band 71-175 Hz with spatial resolution 1 mm in the area of 80×80×80 cubic millimeters. The comparison of the photo and functional structure shows correspondence of both objects in their size and form.

The disclosed embodiments can be used in various ways. For example, the anatomical structure determined by the various embodiments can be used as part of treating a patient. In one scenario, a medical device can administer therapeutic agents at targeted sites. For example, an area of the brain the processes a signal input can be targeted for treatment. The processing pathway through the brain can be identified and then one or more therapeutic agents can be administered to part or the entire processing pathway. The therapeutic agents can also be applied to particular anatomical structures of an organ such as the brain or hear based upon the information from the disclosed analytical techniques. For example, a particular area of a structure can be identified and the therapeutic agent can be applied to just the particular area, allowing for a focused application of the therapeutic agent. In another scenario, the disclosed embodiments can be coupled with a neurostimulation device. The neurostimulation device can work in a closed loop fashion and can be guided based upon a determined processing pathway or anatomical structure. In this scenario, the combination of the neurostimulation device and the disclosed embodiments can be used to treat various disorders. The information produced by the disclosed embodiments can also be used to signal an alarm when certain clinical conditions have been met. As one example, an anatomical structure of a patient can be analyzed to create a baseline of the anatomical structure. The anatomical structure can continue to be monitored, either continuously and on a schedule. The latest anatomical structure can be compared to the baseline to determine if the patient has had a change in condition that requires attention. An alarm can be raised if this is the case.

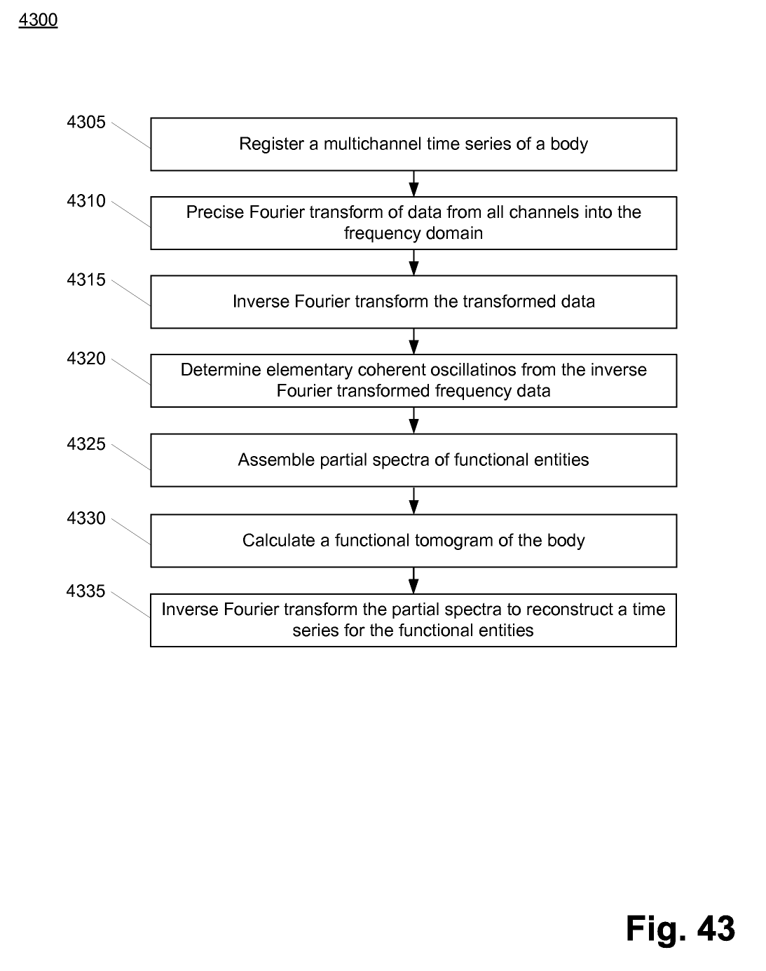

FIG. 43 is a flow diagram of a process 4300 for generating a functional tomographical map in accordance with an illustrative implementation. The process 4300 can be implemented on a computing device. In one implementation, the process 4300 is encoded on a computer-readable medium that contains instructions that, when executed by a computing device, cause the computing device to perform operations of process 4300.

A space is monitored over a period of time across multiple channels, e.g., using multiple sensors. For example, the brain of a subject can be monitored using magnetic field sensors. The data from the multiple channels is used for the registration of a multichannel time series (4305). A precise Fourier transform of the data from all channels is done (4310). The transform translates the data into the frequency domain. An Inverse Fourier transform is done for each frequency in the frequency domain of the transformed data. (4315). Elementary coherent oscillations from the inverse Fourier transformed data are determined (4320). Partial spectra for the functional entities are assembled. (4325). The partial spectra comprises frequencies with similar patterns of the elementary coherent oscillations. A functional tomogram of the body is calculated from the distribution in space and directions of the partial spectra (4330). The partial spectrum is inverse Fourier transformed to reconstruct a time series for the functional entities. (4335).

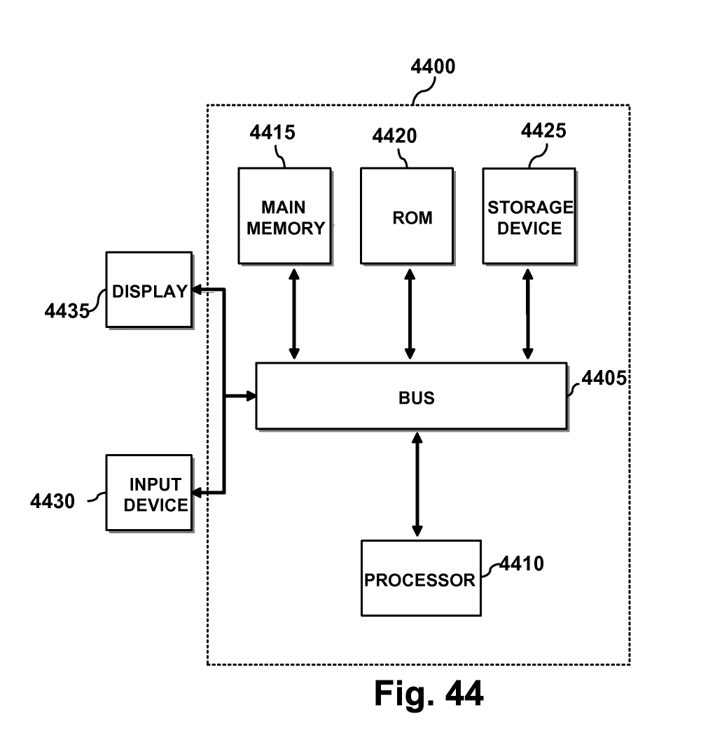

FIG. 44 is a block diagram of a computer system in accordance with an illustrative implementation. The computing system 4400 includes a bus 4405 or other communication component for communicating information and a processor 4410 or processing circuit coupled to the bus 4405 for processing information. The computing system 4400 can also include one or more processors 4410 or processing circuits coupled to the bus for processing information. The computing system 4400 also includes main memory 4415, such as a random access memory (RAM) or other dynamic storage device, coupled to the bus 4405 for storing information, and instructions to be executed by the processor 4410. Main memory 4415 can also be used for storing position information, temporary variables, or other intermediate information during execution of instructions by the processor 4410. The computing system 4400 may further include a read only memory (ROM) 4420 or other static storage device coupled to the bus 4405 for storing static information and instructions for the processor 4410. A storage device 4425, such as a solid state device, magnetic disk or optical disk, is coupled to the bus 4405 for persistently storing information and instructions.

The computing system 4400 may be coupled via the bus 4405 to a display 4435, such as a liquid crystal display, or active matrix display, for displaying information to a user. An input device 4430, such as a keyboard including alphanumeric and other keys, may be coupled to the bus 4405 for communicating information and command selections to the processor 4410. In another implementation, the input device 4430 has a touch screen display 4435. The input device 4430 can include a cursor control, such as a mouse, a trackball, or cursor direction keys, for communicating direction information and command selections to the processor 4410 and for controlling cursor movement on the display 4435.

According to various implementations, the processes described herein can be implemented by the computing system 4400 in response to the processor 4410 executing an arrangement of instructions contained in main memory 4415. Such instructions can be read into main memory 4415 from another computer-readable medium, such as the storage device 4425. Execution of the arrangement of instructions contained in main memory 4415 causes the computing system 4400 to perform the illustrative processes described herein. One or more processors in a multi-processing arrangement may also be employed to execute the instructions contained in main memory 4415. In alternative implementations, hard-wired circuitry may be used in place of or in combination with software instructions to effect illustrative implementations. Thus, implementations are not limited to any specific combination of hardware circuitry and software.

Although an example computing system has been described in FIG. 44, implementations described in this specification can be implemented in other types of digital electronic circuitry, or in computer software, firmware, or hardware, including the structures disclosed in this specification and their structural equivalents, or in combinations of one or more of them.

Implementations described in this specification can be implemented in digital electronic circuitry, or in computer software, firmware, or hardware, including the structures disclosed in this specification and their structural equivalents, or in combinations of one or more of them. The implementations described in this specification can be implemented as one or more computer programs, i.e., one or more modules of computer program instructions, encoded on one or more computer storage media for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate components or media (e.g., multiple CDs, disks, or other storage devices). Accordingly, the computer storage medium is both tangible and non-transitory.

The operations described in this specification can be performed by a data processing apparatus on data stored on one or more computer-readable storage devices or received from other sources.

The term “data processing apparatus” or “computing device” encompasses all kinds of apparatus, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method comprising:
registering a multichannel time series from data collected from a plurality of channels monitoring a body, wherein each channel corresponds to one of one or more sensors, and wherein the body comprises a plurality of functional entities;
precise Fourier transforming, using a processor, the data from all channels to frequency data in a frequency domain;
inverse Fourier transforming, using the processor, the frequency data for each frequency in the frequency domain;
determining, using the processor, elementary coherent oscillations from the inverse Fourier transformed frequency data;
assembling, using the processor, partial spectra of the functional entities comprising frequencies with similar patterns of the elementary coherent oscillations;
calculating a functional tomogram of the body from a distribution in space of the energy or dominant directions of the sources producing the partial spectra; and
inverse Fourier transforming, using the processor, the partial spectra of the functional entities to reconstruct a time series for the functional entities producing the similar patterns of the elementary coherent oscillations.

2. The method of claim 1, further comprising tuning the precise Fourier transform to a selected frequency.

3. The method of claim 1, wherein assembling the partial spectra of one of the functional entities comprises:
receiving a trial normalized pattern associated with the one of the functional entities;
determining a difference between the trial normalized pattern and each of the elementary coherent oscillations;
filtering elementary coherent oscillations based upon a predetermined pattern accuracy amount; and
determining, using the processor, a partial spectrum of the functional entity corresponding to the trial pattern based upon the unfiltered elementary coherent oscillations.

4. The method of claim 1, further comprising:
determining the functional entity is interference; and;
subtracting the partial spectrum from the elementary coherent oscillations.

5. The method of claim 1, further comprising determining the functional entity is not interference.

6. The method of claim 5, further comprising:
selecting a restored time; and
localizing activity of the functional entity for the selected frequencies at the restored time.

7. The method of claim 1, wherein the inverse Fourier transforming comprises determining a location of one or more dipoles.

8. The method of claim 1, further comprising:
dividing the body into a plurality of blocks, wherein each block has one or more dipoles in the center of the block; and
calculating the functional tomogram based upon the distribution of the energy produced by sources in the plurality of blocks.

9. The method of claim 1, further comprising:
determining an energy for each of two or more directions from a dipole;
determining a direction of the functional entity from the dipole based upon the energy of the two or more directions from the dipole.

10. The method of claim 9, further comprising:
dividing the body into a plurality of blocks, wherein each block has one or more dipoles in the center of the block; and
calculating the functional tomogram based upon the distribution of the dominant directions of the sources in the plurality of blocks.

11. The method of claim 1, wherein the sensors are magnetic field sensors.

12. The method of claim 11, further comprising:
determining if localized activity is near borders of one or more of the magnetic field sensors; and
determining the localized activity is noise based upon the localized activity being located near borders of one or more of the magnetic field sensors.

13. A system comprising:
one or more processors configured to:
register a multichannel time series from data collected from a plurality of channels monitoring a body, wherein each channel corresponds to one of one or more sensors, and wherein the body comprises a plurality of functional entities;
precise Fourier transform the data from all channels to frequency data in a frequency domain;
inverse Fourier transform the frequency data for each frequency in the frequency domain;
determine elementary coherent oscillations from the inverse Fourier transformed frequency data;
assemble partial spectra of the functional entities comprising frequencies with similar patterns of the elementary coherent oscillations;
calculate a functional tomogram of the body from a distribution in space of the energy or dominant directions of the sources producing the partial spectra; and
inverse Fourier transform the partial spectra of the functional entities to reconstruct a time series for the functional entities producing the similar patterns of the elementary coherent oscillations.

14. The system of claim 13, wherein the one or more processors are further configured to:
determine the functional entity is interference; and
subtract the partial spectrum from the elementary coherent oscillations.

15. The system of claim 13, wherein the one or more processors are further configured to determine the functional entity is not interference.

16. The system of claim 15, wherein the one or more processors are further configured to:
select a restored time; and
localize activity of the functional entity for the selected frequencies at the restored time.

17. The system of claim 13, wherein the one or more processors are further configured to:
divide the body into a plurality of blocks, wherein each block has one or more dipoles in the center of the block; and
calculate the functional tomogram based upon the distribution of the calculated energy or dominant directions of the dipoles in the plurality of blocks

18. A non-transitory computer readable medium having instructions stored thereon that, when executed by a computing device, cause the computing device to perform operations comprising:
registering a multichannel time series from data collected from a plurality of channels monitoring a body, wherein each channel corresponds to one of one or more sensors, and wherein the body comprises a plurality of functional entities;
precise Fourier transforming the data from all channels to frequency data in a frequency domain;
inverse Fourier transforming the frequency data for each frequency in the frequency domain;
determining elementary coherent oscillations from the inverse Fourier transformed frequency data;
assembling partial spectra of the functional entities comprising frequencies with similar patterns of the elementary coherent oscillations;
calculating a functional tomogram of the body from a distribution in space of the energy or dominant directions of the sources producing the partial spectra; and
inverse Fourier transforming the partial spectra of the functional entities to reconstruct a time series for the functional entities producing the similar patterns of the elementary coherent oscillations.

19. The non-transitory computer readable medium of claim 18, wherein the operations further comprise:
determining the functional entity is interference; and;
subtracting the partial spectrum from the elementary coherent oscillations.

20. The non-transitory computer readable medium of claim 18, wherein the operations further comprise:
dividing the body into a plurality of blocks, wherein each block has one or more dipoles in the center of the block; and
calculating the functional tomogram, based upon the distribution of the calculated energy or dominant directions of the dipoles in the plurality of blocks.
